(19) United States
(12) Patent Application Publication
(10) Pub. No.: US 2024/0263228 A1
(43) Pub. Date: Aug. 8, 2024

(54) Next-Generation Volumetric in Situ Sequencing

(71) Applicant: The Board of Trustees of the Leland Stanford Junior University, Stanford, CA (US)

(72) Inventors: Karl A. Deisseroth, Stanford, CA (US); Ethan B. Richman, Palo Alto, CA (US)

(21) Appl. No.: 18/561,259

(22) PCT Filed: May 20, 2022
(86) PCT No.: PCT/US2022/030370

§ 371 Date: Nov. 15, 2023

Related U.S. Application Data
(60) Provisional application No. 63/191,455, filed on May 21, 2021.

(51) Int. Cl. C12Q 1/6874; C12Q 1/6841

(52) U.S. Cl. CPC C12Q 1/6874; C12Q 1/6841

(57) ABSTRACT

Provided herein are devices, methods, and systems for next-generation volumetric in situ sequencing of nucleic acids in cells in intact tissue. In particular, methods are provided for improving robustness across sample types, including for thin and thick tissue volumes, and increasing the efficiency and specificity of target labeling for volumetric combinatorial in situ sequencing.

### BACKGROUND OF THE INVENTION

Biological samples contain complex and heterogenous genetic information spanning the length scales of individual cells and whole tissues. Spatial patterns of nucleic acids within a cell may reveal properties and abnormalities of cellular function; cumulative distributions of RNA expression may define a cell type or function; and systematic variation in the locations of cell types within a tissue may define tissue function. The combination of anatomical connectivity information encoded in nucleic acids and tissue-wide cell type distributions may span many sections of tissue. Techniques for in situ nucleic acid sequencing must therefore be able to bridge resolutions as small as individual molecules and as large as entire brains. Efficiently collecting and recording this information across orders-of-magnitude differences in lengths requires novel inventions to enhance the robustness, rapidity, automated-, and high throughput-nature of in situ sequencing techniques.

### SUMMARY OF THE INVENTION

Provided herein are devices, methods, and systems for next-generation volumetric in situ sequencing of nucleic acids in cells in intact tissue. In particular, methods are provided for improving robustness across sample types, including for thin and thick tissue volumes, and increasing the efficiency and specificity of target labeling for volumetric sequential and combinatorial in situ sequencing.

In one aspect, a method for in situ gene sequencing of a target nucleic acid in a cell in an intact tissue is provided, the method comprising: (a) contacting a fixed and permeabilized intact tissue with at least a pair of oligonucleotide primers under conditions to allow for specific hybridization, wherein the pair of primers comprise a first oligonucleotide and a second oligonucleotide; wherein each of the first oligonucleotide and the second oligonucleotide comprises a first complementarity region, a second complementarity region sequence, and a third complementarity region; wherein the second oligonucleotide further comprises a barcode sequence; wherein the first complementarity region of the first oligonucleotide is complementary to a first portion of the target nucleic acid, wherein the second complementarity region of the first oligonucleotide is complementary to the first complementarity region of the second oligonucleotide, wherein the third complementarity region of the first oligonucleotide is complementary to the third complementarity region of the second oligonucleotide, wherein the second complementary region of the second oligonucleotide is complementary to a second portion of the target nucleic acid, wherein the first portion of the target nucleic is adjacent to the second portion of the target nucleic acid, wherein the second complementarity region of the first oligonucleotide comprises a first portion of a unique matching sequence and the third complementarity region of the first oligonucleotide comprises a second portion of the unique matching sequence, and the first complementarity region of the second oligonucleotide comprises a sequence that is complementary to the first portion of the unique matching sequence, and the third complementarity region of the second oligonucleotide comprises a sequence that is complementary to the second portion of the unique matching sequence; (b) adding ligase to ligate the second oligonucleotide and generate a closed nucleic acid circle; (c) performing rolling circle amplification in the presence of a nucleic acid molecule, wherein the performing comprises using the second oligonucleotide as a template and the first oligonucleotide as a primer for a polymerase to form one or more amplicons; (d) embedding the one or more amplicons in the presence of hydrogel subunits to form one or more hydrogel-embedded amplicons; (e) contacting the one or more hydrogel-embedded amplicons having the barcode sequence with a set of sequencing primers under conditions to allow for ligation, wherein the set of sequencing primers comprises a third oligonucleotide configured to decode bases and a fourth oligonucleotide configured to convert decoded bases into a signal, wherein the ligation only occurs when both the third oligonucleotide and the fourth oligonucleotide are complementary to adjacent sequences of the same amplicon; (f) reiterating step (e); and (g) imaging the one or more hydrogel-embedded amplicons to determine in situ a gene sequence of the target nucleic acid in the cell in the intact tissue.

In certain embodiments, the method further comprises contacting the fixed and permeabilized intact tissue with a gel adaptor oligonucleotide that binds to the first oligonucleotide, wherein the gel adaptor oligonucleotide comprises a nucleotide modification at the 5′ end or the 3′ end that links the gel adapter to the hydrogel during gelation. In some embodiments, the modification comprises an acrydite group. In some embodiments, the first oligonucleotide further comprises a common binding site for the gel adaptor oligonucleotide. In some embodiments, the common binding site for the gel adaptor oligonucleotide is adjacent to the first complementarity region of the first oligonucleotide.

In certain embodiments, the method further comprises contacting the fixed and permeabilized intact tissue with an oligonucleotide probe for detection and condensing of the amplicons, wherein the oligonucleotide probe binds to the second oligonucleotide. In some embodiments, the second oligonucleotide further comprises a common binding site for the oligonucleotide probe for detection and condensing of the amplicons. In some embodiments, the common binding site for the oligonucleotide probe for detection and condensing of the amplicons is adjacent to the second complementarity region or adjacent to the sequence that is complementary to the second half of the unique matching sequence of the second oligonucleotide. In some embodiments, the oligonucleotide probe comprises a unique sequence for detecting an amplicon of a probe target of interest and two or more copies of a sequence complementary to a common sequence on the amplicons. In some embodiments, the oligonucleotide probe further comprises a nucleotide modification at the 5′ end or the 3′ end such that the first oligonucleotide probe is linked to the hydrogel during gelation. In some embodiments, the modification comprises an acrydite group.

In certain embodiments, the method further comprise barcoding a cell by contacting the cell with: i) a first probe comprising a 5′-amine modification or a 5′-biotin modification, a common gel adaptor complementary sequence that hybridizes with the gel adaptor oligonucleotide, and a unique barcode sequence; and ii) a second probe comprising a first sequence that is complementary to a first portion of the unique barcode sequence and a second sequence that is complementary to a second portion of the unique barcode sequence, wherein the first sequence and the second sequence flank a sequencing encoding sequence, wherein hybridization of the first probe and the second probe results in formation of a barcoding complex comprising the first probe and the second probe. In some embodiments, the second probe is a padlock probe.

In certain embodiments, the first portion of the target nucleic acid and the second portion of the target nucleic acid have about the same melting temperature.

In certain embodiments, the method further comprises contacting the fixed and permeabilized intact tissue with an mRNA retention oligonucleotide, wherein the mRNA retention oligonucleotide comprises: a nucleotide modification at the 5′ end or the 3′ end, wherein the mRNA retention oligonucleotide is linked to the hydrogel during gelation; a poly-T tail that hybridizes to a poly-A tail of an mRNA, wherein hybridization of the poly-T tail of the mRNA retention oligonucleotide to the poly-A tail of the mRNA retains the mRNA in the hydrogel; and a unique hybridization sequence. In some embodiments, the poly-T tail comprises interleaved locked nucleic acid (LNA) thymine (T) bases. In some embodiments, the method further comprises contacting the fixed and permeabilized intact tissue with a fluorescently labeled probe oligonucleotide that selectively binds to the unique hybridization sequence of the mRNA retention oligonucleotide.

In certain embodiments, the unique matching sequence is a randomized sequence.

In certain embodiments, the sequencing is performed with sequential or combinatorial encoding.

In certain embodiments, the method further comprises preincubating the tissue sample with the polymerase for a sufficient time to allow uniform diffusion of the polymerase throughout the tissue before performing the rolling circle amplification.

In certain embodiments, the signal is a fluorescent signal.

In certain embodiments, imaging is performed in the presence of an anti-fade buffer comprising an antioxidant. For example, the anti-fade buffer may comprise, without limitation, trolox (6-hydroxy-2,5,7,8-tetramethylchroman-2-carboxylic acid) and trolox-quinone.

In certain embodiments, the method further comprises removing the signal after imaging by contacting the hydrogel with formamide.

In certain embodiments, the fourth oligonucleotide is covalently linked to a fluorophore by a disulfide bond. In some embodiments, the method further comprises contacting the hydrogel with a reducing agent after said imaging, wherein reduction of the disulfide bond results in cleavage of the fluorophore from the fourth oligonucleotide.

In certain embodiments, ligation of the third oligonucleotide and the fourth oligonucleotide is performed in presence of a polyethylene glycol polymer (e.g., PEG 6000).

In certain embodiments, the oligo-conjugated to the target-binding agent is detected by hybridization of an oligonucleotide containing a fluorophore. In certain embodiments, this signal is removed, after detection, by competitive binding of a displacing oligonucleotide.

In another aspect, a method of screening a candidate agent to determine whether the candidate agent modulates gene expression of a nucleic acid in a cell in an intact tissue is provided, the method comprising performing a method described herein to determine the gene sequence of the target nucleic acid in the cell in the intact tissue, and detecting the level of gene expression of the target nucleic acid, wherein an alteration in the level of expression of the target nucleic acid in the presence of the candidate agent relative to the level of expression of the target nucleic acid in the absence of the candidate agent indicates that the candidate agent modulates gene expression of the nucleic acid in the cell in the intact tissue.

In certain embodiments, the detecting comprises performing flow cytometry; sequencing; probe binding and electrochemical detection; pH alteration; catalysis induced by enzymes bound to DNA tags; quantum entanglement; Raman spectroscopy; terahertz wave technology; and/or scanning electron microscopy. In some embodiments, the flow cytometry is mass cytometry or fluorescence-activated flow cytometry. In some embodiments, the detecting comprises performing microscopy, scanning mass spectrometry, or other imaging techniques. In some embodiments, the detecting comprises detecting a signal such as a fluorescent signal.

In another aspect, a system is provided, the system comprising a fluidics device, and a processor unit configured to perform a method described herein for in situ gene sequencing of a target nucleic acid in a cell in an intact tissue.

In certain embodiments, the system further comprises an imaging chamber.

In certain embodiments, the system further comprises a pump.

### BRIEF DESCRIPTION OF THE DRAWINGS

FIGS. 1A-1D. Next-gen STARmap2 signal across a thick section (200 um) mouse brain tissue sample. Maintaining the section floating throughout the sample preparation, combined with dextran in the hybridization solution, PEG in the ligation solution, and preincubation with inactive RCA polymerase solution result in consistent, high efficiency and uniform labeling throughout the sample Z axis.

FIG. 2. Efficient, high signal-to-noise ratio (SNR) labeling by STARmap2 in a postmortem human brain tissue sample. A single read out channel (488) of four acquired channels is shown in a single optical plane, from the first combinatorial round of sequencing. Dashed box highlights an individual cell in the tissue. Punctate signals are amplicons.

FIG. 3. Workflow for STARmap2 in which samples are maintained floating throughout hybridization, clearing, ligation, and RCA reactions, before they are reattached in preparation for sequencing.

FIG. 4. Combinatorial chemistry sequencing of 200 genes in a 150 um thick mouse brain section. Data shown single optical planes from a single combinatorial round of labeling. Left, a single XY optical plane. Top right, a ZY optical plane showing consistent labeling efficiency across the Z axis with high SNR throughout. Bottom right, detail for several cells in a single XY plane.

FIGS. 5A-5B. Effect of incorporating unique sequence at probe circularization junction. (A, B) Samples were prepared with a common set of STARmap2 probes against Actb, plus 384 probes targeting random (exogenous) sequences not contained within the samples. (FIG. 5A) Sample labeled with 384 probes targeting random sequence contained unique (across probes) sequences at their circularization junction. Unique sequences were generated by randomizing sequence and filtering by cross-sequence similarity and melting temperature. (FIG. 5B) Sample labeled with 384 probes targeting the same set of random (exogenous) sequence as in (FIG. 5A), except probes contain constant sequence at the circularization junction, as in the original SNAIL probe design. Bottom right inset of (FIG. 5A) and (FIG. 5B) showing detail on space between cells. Dashed line boxes, inset locations. Samples prepared side by side from neighboring tissues sections. Images acquired under 20× magnification from the single brightest plane, with equal imaging and contrast settings.

FIG. 6. Overview of STARmap2 probe modifications (excluding detail on sequences used for improved sequencing chemistries).

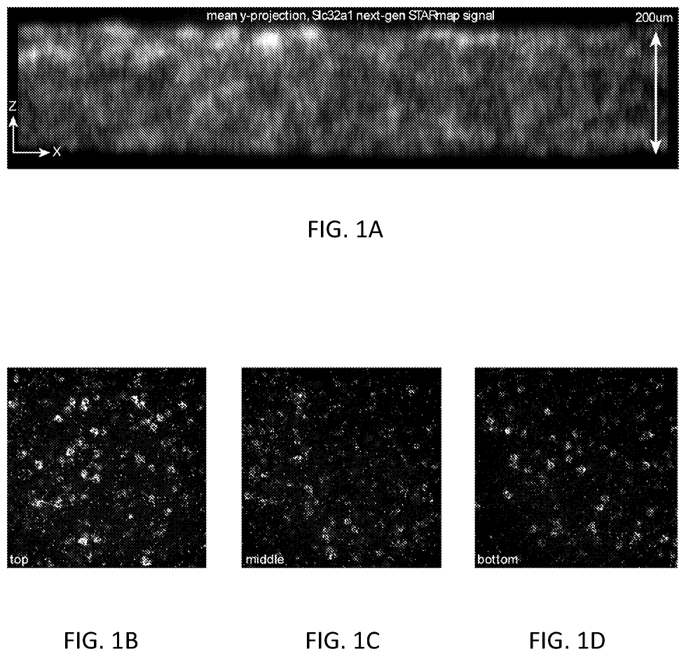

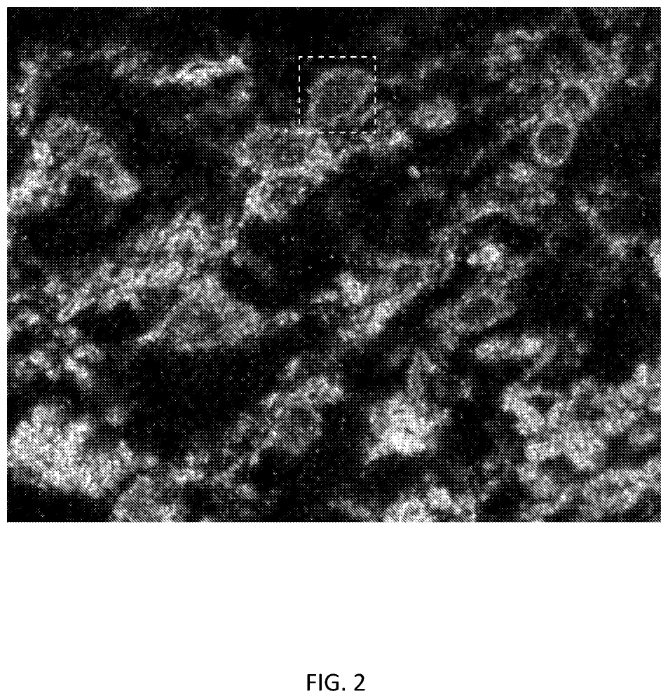

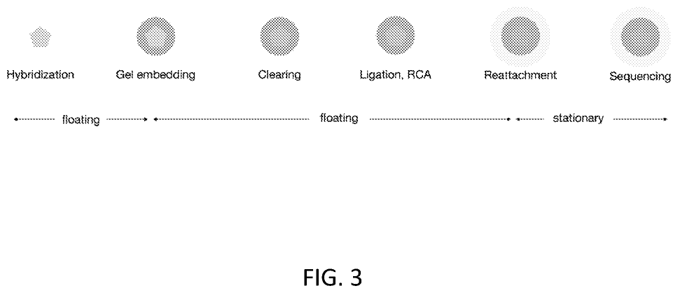

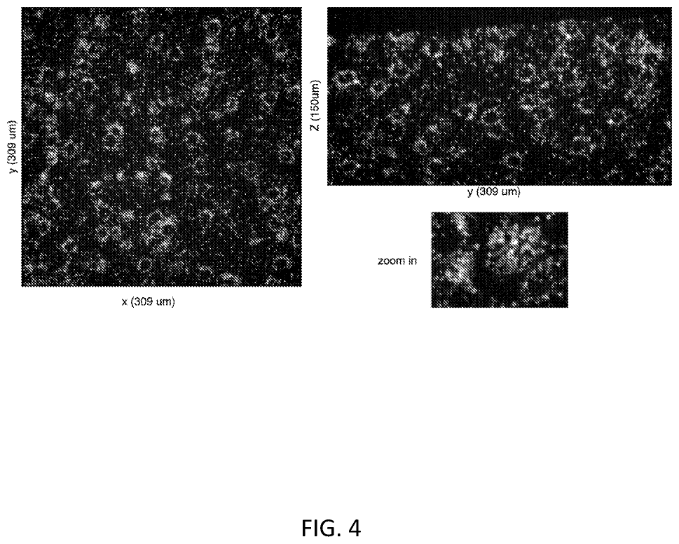

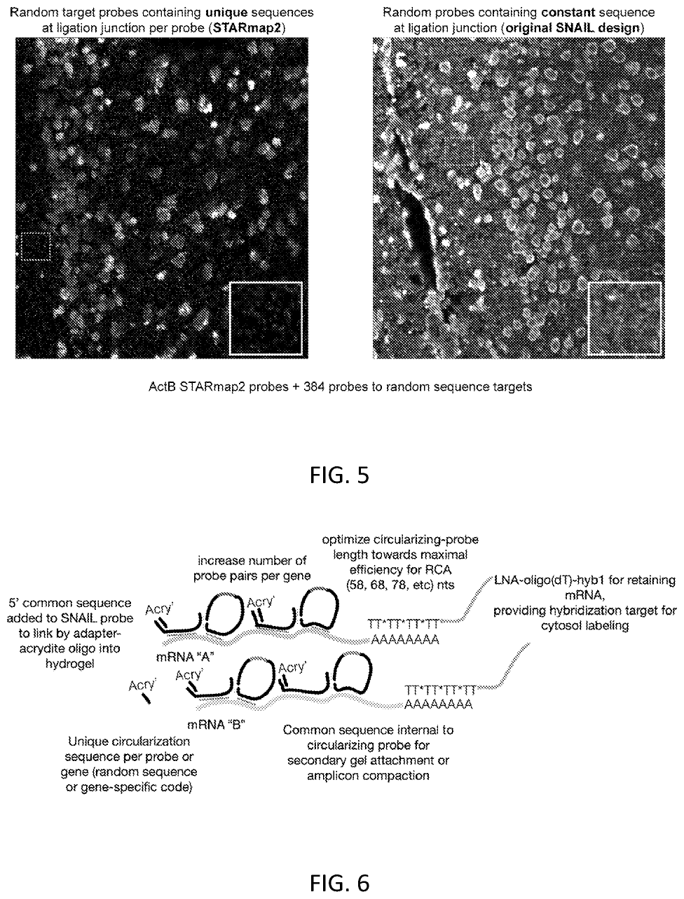

### DETAILED DESCRIPTION OF THE INVENTION

Provided herein are devices, methods, and systems for next-generation volumetric in situ sequencing of nucleic acids in cells in intact tissue. In particular, methods are provided for improving robustness across sample types, including for thin and thick tissue volumes, and increasing the efficiency and specificity of target labeling for volumetric combinatorial in situ sequencing.

Before the present devices, methods, and systems are described, it is to be understood that this invention is not limited to particular methods or compositions described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

It must be noted that as used herein and in the appended claims, the singular forms “a”, “an”, and “the” include plural referents unless the context clearly dictates otherwise. Thus, for example, reference to “a cell” includes a plurality of such cells and reference to “the peptide” includes reference to one or more peptides and equivalents thereof, e.g. oligopeptides or polypeptides known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### Definitions

The term “about”, particularly in reference to a given quantity, is meant to encompass deviations of plus or minus five percent.

The terms “peptide”, “oligopeptide”, “polypeptide”, and “protein” are used interchangeably herein to refer to a polymer of amino acid residues. The terms also apply to amino acid polymers in which one or more amino acid residue is an artificial chemical mimetic of a corresponding naturally occurring amino acid, as well as to naturally occurring amino acid polymers and non-naturally occurring amino acid polymers. Both full-length proteins and fragments thereof are encompassed by the definition. The terms also include post-expression modifications of the polypeptide, for example, phosphorylation, glycosylation, acetylation, hydroxylation, oxidation, and the like as well as chemically or biochemically modified or derivatized amino acids and polypeptides having modified peptide backbones. The terms also include fusion proteins, including, but not limited to, fusion proteins with a heterologous amino acid sequence, fusions with heterologous and homologous leader sequences, with or without N-terminal methionine residues; immunologically tagged proteins; and the like. The terms include polypeptides including one or more of a fatty acid moiety, a lipid moiety, a sugar moiety, and a carbohydrate moiety.

As used herein, the term “target nucleic acid” is any polynucleotide nucleic acid molecule (e.g., DNA molecule; RNA molecule, modified nucleic acid, etc.) present in a single cell. In some embodiments, the target nucleic acid is a coding RNA (e.g., mRNA). In some embodiments, the target nucleic acid is a non-coding RNA (e.g., tRNA, rRNA, microRNA (miRNA), mature miRNA, immature miRNA; etc.). In some embodiments, the target nucleic acid is a splice variant of an RNA molecule (e.g., mRNA, pre-mRNA, etc.) in the context of a cell. A suitable target nucleic acid can therefore be an unspliced RNA (e.g., pre-mRNA, mRNA), a partially spliced RNA, or a fully spliced RNA, etc. Target nucleic acids of interest may be variably expressed, i.e. have a differing abundance, within a cell population, wherein the methods of the invention allow profiling and comparison of the expression levels of nucleic acids, including without limitation RNA transcripts, in individual cells. A target nucleic acid can also be a DNA molecule, e.g. a denatured genomic, viral, plasmid, etc. For example, the methods can be used to detect copy number variants, e.g. in a cancer cell population in which a target nucleic acid is present at different abundance in the genome of cells in the population; a virus-infected cells to determine the virus load and kinetics, and the like.

The terms “oligonucleotide,” “polynucleotide,” and “nucleic acid molecule”, used interchangeably herein, refer to polymeric forms of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer including purine and pyrimidine bases or other natural, chemically or biochemically modified, non-natural, or derivatized nucleotide bases. The backbone of the polynucleotide can include sugars and phosphate groups (as may typically be found in RNA or DNA), or modified or substituted sugar or phosphate groups. Alternatively, the backbone of the polynucleotide can include a polymer of synthetic subunits such as phosphoramidites, and/or phosphorothioates, and thus can be an oligodeoxynucleoside phosphoramidate or a mixed phosphoramidate-phosphodiester oligomer. Peyrottes et al. (1996) Nucl. Acids Res. 24:1841-1848; Chaturvedi et al. (1996) Nucl. Acids Res. 24:2318-2323. The polynucleotide may include one or more L-nucleosides. A polynucleotide may include modified nucleotides, such as methylated nucleotides and nucleotide analogs, uracyl, other sugars, and linking groups such as fluororibose and thioate, and nucleotide branches. The sequence of nucleotides may be interrupted by non-nucleotide components. A polynucleotide may be modified to include N3′-P5′ (NP) phosphoramidate, morpholino phosphorociamidate (MF), locked nucleic acid (LNA), 2′-O-methoxyethyl (MOE), or 2′-fluoro, arabino-nucleic acid (FANA), which can enhance the resistance of the polynucleotide to nuclease degradation (see, e.g., Faria et al. (2001) Nature Biotechnol. 19:40-44; Toulme (2001) Nature Biotechnol. 19:17-18). A polynucleotide may be further modified after polymerization, such as by conjugation with a labeling component. Other types of modifications included in this definition are caps, substitution of one or more of the naturally occurring nucleotides with an analog, and introduction of means for attaching the polynucleotide to proteins, metal ions, labeling components, other polynucleotides, or a solid support. Immunomodulatory nucleic acid molecules can be provided in various formulations, e.g., in association with liposomes, microencapsulated, etc., as described in more detail herein. A polynucleotide used in amplification is generally single-stranded for maximum efficiency in amplification, but may alternatively be double-stranded. If double-stranded, the polynucleotide can first be treated to separate its strands before being used to prepare extension products. This denaturation step is typically affected by heat, but may alternatively be carried out using alkali, followed by neutralization.

By “isolated” is meant, when referring to a protein, polypeptide, or peptide, that the indicated molecule is separate and discrete from the whole organism with which the molecule is found in nature or is present in the substantial absence of other biological macro molecules of the same type. The term “isolated” with respect to a polynucleotide is a nucleic acid molecule devoid, in whole or part, of sequences normally associated with it in nature; or a sequence, as it exists in nature, but having heterologous sequences in association therewith; or a molecule disassociated from the chromosome.

The terms “individual”, “subject”, “host”, and “patient”, are used interchangeably herein and refer to invertebrates and vertebrates including, but not limited to, arthropods (e.g., insects, crustaceans, arachnids), cephalopods (e.g., octopuses, squids), amphibians (e.g., frogs, salamanders, caecilians), fish, reptiles (e.g., turtles, crocodilians, snakes, amphisbaenians, lizards, tuatara), mammals, including human and non-human mammals such as non-human primates, including chimpanzees and other apes and monkey species; laboratory animals such as mice, rats, rabbits, hamsters, guinea pigs, and chinchillas; domestic animals such as dogs and cats; farm animals such as sheep, goats, pigs, horses and cows; and birds such as domestic, wild and game birds, including chickens, turkeys and other gallinaceous birds, ducks, and geese. In some cases, the methods of the invention find use in experimental animals, in veterinary application, and in the development of animal models for disease, including, but not limited to, rodents including mice, rats, and hamsters; primates, and transgenic animals.

### Methods

The methods disclosed herein include the use of a modified Spatially-resolved Transcript Amplicon Readout Mapping (STARmap) technique, termed “STARmap2”. For a description of the original STARmap technique, see, e.g., International Patent Application Publication No. WO2019/199579A1 and Wang et al. (2018) Science 361(6400):eaat5691; herein incorporated by reference in their entireties. Like STARmap, STARmap2 utilizes an image-based in situ nucleic acid (DNA and/or RNA) sequencing technology using a sequencing-by-ligation process, specific signal amplification, hydrogel-tissue chemistry to turn biological tissue into a transparent sequencing chip, and associated data analysis pipelines to spatially-resolve highly-multiplexed gene detection at a subcellular and cellular level. STARmap2 adds improvements in probe design to reduce background labeling, use of adaptor oligonucleotides for better labeling and retention of nucleic acid targets in gels, reduces imaging cycling time, and provides the capability to perform either forward or backward sequencing with high efficiency combinatorial barcode reads.

As summarized above, the methods disclosed herein include a method for in situ gene sequencing of a target nucleic acid in a cell in an intact tissue, the method including: (a) contacting a fixed and permeabilized intact tissue with at least a pair of oligonucleotide primers under conditions to allow for specific hybridization, wherein the pair of primers comprise a first oligonucleotide and a second oligonucleotide; wherein each of the first oligonucleotide and the second oligonucleotide comprises a first complementarity region, a second complementarity region sequence, and a third complementarity region; wherein the second oligonucleotide further comprises a barcode sequence; wherein the first complementarity region of the first oligonucleotide is complementary to a first portion of the target nucleic acid, wherein the second complementarity region of the first oligonucleotide is complementary to the first complementarity region of the second oligonucleotide, wherein the third complementarity region of the first oligonucleotide is complementary to the third complementarity region of the second oligonucleotide, wherein the second complementary region of the second oligonucleotide is complementary to a second portion of the target nucleic acid, wherein the first portion of the target nucleic is adjacent to the second portion of the target nucleic acid, wherein the second complementarity region of the first oligonucleotide comprises a first portion of a unique matching sequence and the third complementarity region of the first oligonucleotide comprises a second portion of the unique matching sequence, and the first complementarity region of the second oligonucleotide comprises a sequence that is complementary to the first portion of the unique matching sequence, and the third complementarity region of the second oligonucleotide comprises a sequence that is complementary to the second portion of the unique matching sequence; (b) adding ligase to ligate the second oligonucleotide and generate a closed nucleic acid circle; (c) performing rolling circle amplification in the presence of a nucleic acid molecule, wherein the performing comprises using the second oligonucleotide as a template and the first oligonucleotide as a primer for a polymerase to form one or more amplicons; (d) embedding the one or more amplicons in the presence of hydrogel subunits to form one or more hydrogel-embedded amplicons; (e) contacting the one or more hydrogel-embedded amplicons having the barcode sequence with a set of sequencing primers under conditions to allow for ligation, wherein the set of sequencing primers comprises a third oligonucleotide configured to decode bases and a fourth oligonucleotide configured to convert decoded bases into a signal, wherein the ligation only occurs when both the third oligonucleotide and the fourth oligonucleotide are complementary to adjacent sequences of the same amplicon; (f) reiterating step (e); and (g) imaging the one or more hydrogel-embedded amplicons to determine in situ a gene sequence of the target nucleic acid in the cell in the intact tissue.

In certain embodiments, the method further comprises contacting the fixed and permeabilized intact tissue with a gel adaptor oligonucleotide that binds to the first oligonucleotide, wherein the gel adaptor oligonucleotide comprises a nucleotide modification at the 5′ end or the 3′ end that links the gel adapter to the hydrogel during gelation. In some embodiments, the modification comprises an acrydite group. In some embodiments, the first oligonucleotide further comprises a common binding site for the gel adaptor oligonucleotide. In some embodiments, the common binding site for the gel adaptor oligonucleotide is adjacent to the first complementarity region of the first oligonucleotide.

In certain embodiments, the method further comprises contacting the fixed and permeabilized intact tissue with an oligonucleotide probe for detection and condensing of the amplicons, wherein the oligonucleotide probe binds to the second oligonucleotide. In some embodiments, the second oligonucleotide further comprises a common binding site for the oligonucleotide probe for detection and condensing of the amplicons. In some embodiments, the common binding site for the oligonucleotide probe for detection and condensing of the amplicons is adjacent to the second complementarity region or adjacent to the sequence that is complementary to the second half of the unique matching sequence of the second oligonucleotide. In some embodiments, the oligonucleotide probe comprises a unique sequence for detecting an amplicon of a probe target of interest and two or more copies of a sequence complementary to a common sequence on the amplicons. In some embodiments, the oligonucleotide probe further comprises a nucleotide modification at the 5′ end or the 3′ end such that the first oligonucleotide probe is linked to the hydrogel during gelation. In some embodiments, the modification comprises an acrydite group.

In certain embodiments, the method further comprise barcoding a cell by contacting the cell with: i) a first probe comprising a 5′-amine modification or a 5′-biotin modification, a common gel adaptor complementary sequence that hybridizes with the gel adaptor oligonucleotide, and a unique barcode sequence; and ii) a second probe comprising a first sequence that is complementary to a first portion of the unique barcode sequence and a second sequence that is complementary to ae second portion of the unique barcode sequence, wherein the first sequence and the second sequence flank a sequencing encoding sequence, wherein hybridization of the first probe and the second probe results in formation of a barcoding complex comprising the first probe and the second probe. In some embodiments, the second probe is a padlock probe.

In certain embodiments, the method further comprises contacting the fixed and permeabilized intact tissue with an mRNA retention oligonucleotide, wherein the mRNA retention oligonucleotide comprises: a nucleotide modification at the 5′ end or the 3′ end, wherein the mRNA retention oligonucleotide is linked to the hydrogel during gelation; a poly-T tail that hybridizes to a poly-A tail of an mRNA, wherein hybridization of the poly-T tail of the mRNA retention oligonucleotide to the poly-A tail of the mRNA retains the mRNA in the hydrogel; and a unique hybridization sequence. In some embodiments, the poly-T tail comprises interleaved locked nucleic acid (LNA) thymine (T) bases. In certain embodiments, the method further comprises contacting the fixed and permeabilized intact tissue with a fluorescently labeled probe oligonucleotide that selectively binds to the unique hybridization sequence of the mRNA retention oligonucleotide.

The methods disclosed herein also provide for a method of screening a candidate agent to determine whether the candidate agent modulates gene expression of a nucleic acid in a cell in an intact tissue by performing a method described herein to determine the gene sequence of the target nucleic acid in the cell in the intact tissue, and detecting the level of gene expression of the target nucleic acid, wherein an alteration in the level of expression of the target nucleic acid in the presence of the candidate agent relative to the level of expression of the target nucleic acid in the absence of the candidate agent indicates that the candidate agent modulates gene expression of the nucleic acid in the cell in the intact tissue.

In certain aspects, the methods disclosed herein provide for a faster processing time, higher multiplexity (up to 1000 genes), higher efficiency, higher sensitivity, lower error rate, and more spatially resolved cell types, as compared to existing gene expression analysis tools. In such aspects, the improved hydrogel-tissue chemistry method transforms biological tissue into nucleic acids imprinted with hydrogel compatible with in situ sequencing, improved sequencing-by-ligation techniques (SCAL and SEDAL2) for in situ sequencing with error reduction. In some other aspects, the methods disclosed herein include spatially sequencing (e.g. reagents, chips or services) for biomedical research and clinical diagnostics (e.g. cancer, bacterial infection, viral infection, etc.) with single-cell and/or single-molecule sensitivity.

### Specific Amplification of Nucleic Acids Via Intramolecular Ligation (SNAIL)

In some embodiments, one component of STARmap2 includes an efficient approach for generating cDNA libraries from cellular RNAs in situ, which may be referred to as SNAIL, for Specific Amplification of Nucleic Acids via Intramolecular Ligation. In certain embodiments, the methods of the invention include contacting a fixed and permeabilized intact tissue with at least a pair of oligonucleotide primers under conditions to allow for specific hybridization, wherein the pair of primers includes a first oligonucleotide and a second oligonucleotide.

More generally, the nucleic acid present in a cell of interest in a tissue serves as a scaffold for an assembly of a complex that includes a pair of primers, referred to herein as a first oligonucleotide and a second oligonucleotide. In some embodiments, the contacting the fixed and permeabilized intact tissue includes hybridizing the pair of primers to the same target nucleic acid. In some embodiments, the target nucleic acid is RNA. In such embodiments, the target nucleic acid may be mRNA. In other embodiments, the target nucleic acid is DNA.

As used herein, the terms “hybridize” and “hybridization” refer to the formation of complexes between nucleotide sequences which are sufficiently complementary to form complexes via Watson-Crick base pairing. Where a primer “hybridizes” with target (template), such complexes (or hybrids) are sufficiently stable to serve the priming function required by, e.g., the DNA polymerase to initiate DNA synthesis. It will be appreciated that the hybridizing sequences need not have perfect complementarity to provide stable hybrids. In many situations, stable hybrids will form where fewer than about 10% of the bases are mismatches, ignoring loops of four or more nucleotides. Accordingly, as used herein the term “complementary” refers to an oligonucleotide that forms a stable duplex with its “complement” under assay conditions, generally where there is about 90% or greater homology.

### SNAIL Oligonucleotide Primers

In the subject methods, the SNAIL oligonucleotide primers include at least a first oligonucleotide and a second oligonucleotide; wherein each of the first oligonucleotide and the second oligonucleotide includes a first complementarity region, a second complementarity region, and a third complementarity region; wherein the second oligonucleotide further includes a barcode sequence; wherein the first complementarity region of the first oligonucleotide is complementary to a first portion of the target nucleic acid, wherein the second complementarity region of the first oligonucleotide is complementary to the first complementarity region of the second oligonucleotide, wherein the third complementarity region of the first oligonucleotide is complementary to the third complementarity region of the second oligonucleotide, wherein the second complementary region of the second oligonucleotide is complementary to a second portion of the target nucleic acid, and wherein the first complementarity region of the first oligonucleotide is adjacent to the second complementarity region of the second oligonucleotide. In an alternative embodiment, the second oligonucleotide is a closed circular molecule, and a ligation step is omitted.

The present disclosure provides methods where the contacting a fixed and permeabilized tissue includes hybridizing a plurality of oligonucleotide primers having specificity for different target nucleic acids. In some embodiments, the methods include a plurality of first oligonucleotides, including, but not limited to, 5 or more first oligonucleotides, e.g., 8 or more, 10 or more, 12 or more, 15 or more, 18 or more, 20 or more, 25 or more, 30 or more, 35 or more that hybridize to target nucleotide sequences. In some embodiments, a method of the present disclosure includes a plurality of first oligonucleotides, including, but not limited to, 15 or more first oligonucleotides, e.g., 20 or more, 30 or more, 40 or more, 50 or more, 60 or more, 70 or more, and up to 80 different first oligonucleotides that hybridize to 15 or more, e.g., 20 or more, 30 or more, 40 or more, 50 or more, 60 or more, 70 or more, and up to 80 different target nucleotide sequences. In some embodiments, the methods include a plurality of second oligonucleotides, including, but not limited to, 5 or more second oligonucleotides, e.g., 8 or more, 10 or more, 12 or more, 15 or more, 18 or more, 20 or more, 25 or more, 30 or more, 35 or more. In some embodiments, a method of the present disclosure includes a plurality of second oligonucleotides including, but not limited to, 15 or more second oligonucleotides, e.g., 20 or more, 30 or more, 40 or more, 50 or more, 60 or more, 70 or more, and up to 80 different first oligonucleotides that hybridize to 15 or more, e.g., 20 or more, 30 or more, 40 or more, 50 or more, 60 or more, 70 or more, and up to 80 different target nucleotide sequences. A plurality of oligonucleotide pairs can be used in a reaction, where one or more pairs specifically bind to each target nucleic acid. For example, two primer pairs can be used for one target nucleic acid in order to improve sensitivity and reduce variability. It is also of interest to detect a plurality of different target nucleic acids in a cell, e.g. detecting up to 2, up to 3, up to 4, up to 5, up to 6, up to 7, up to 8, up to 9, up to 10, up to 12, up to 15, up to 18, up to 20, up to 25, up to 30, up to 40 or more distinct target nucleic acids. The primers are typically denatured prior to use, typically by heating to a temperature of at least about 50° C., at least about 60° C., at least about 70° C., at least about 80° C., and up to about 99° C., up to about 95° C., up to about 90° C.

In some embodiments, the primers are denatured by heating before contacting the sample. In certain aspects, the melting temperature (Tₘ) of oligonucleotides is selected to minimize ligation in solution. The “melting temperature” or “Tₘ” of a nucleic acid is defined as the temperature at which half of the helical structure of the nucleic acid is lost due to heating or other dissociation of the hydrogen bonding between base pairs, for example, by acid or alkali treatment, or the like. The Tₘ of a nucleic acid molecule depends on its length and on its base composition. Nucleic acid molecules rich in GC base pairs have a higher Tₘ than those having an abundance of AT base pairs. Separated complementary strands of nucleic acid spontaneously reassociate or anneal to form duplex nucleic acid when the temperature is lowered below the Tₘ. The highest rate of nucleic acid hybridization occurs approximately 25 degrees C. below the Tₘ. The Tₘ may be estimated using the following relationship: Tₘ=69.3+0.41 (GC) % (Marmur et al. (1962) J. Mol. Biol. 5:109-118).

In certain embodiments, the plurality of second oligonucleotides includes a padlock probe. In some embodiments, the probe includes a detectable label that can be measured and quantitated. The terms “label” and “detectable label” refer to a molecule capable of detection, including, but not limited to, radioactive isotopes, fluorescers, chemiluminescers, enzymes, enzyme substrates, enzyme cofactors, enzyme inhibitors, chromophores, dyes, metal ions, metal sols, ligands (e.g., biotin or haptens) and the like. The term “fluorescer” refers to a substance or a portion thereof that is capable of exhibiting fluorescence in the detectable range. Particular examples of labels that may be used with the invention include, but are not limited to phycoerythrin, Alexa dyes, fluorescein, YPet, CyPet, Cascade blue, allophycocyanin, Cy3, Cy5, Cy7, rhodamine, dansyl, umbelliferone, Texas red, luminol, acradimum esters, biotin, green fluorescent protein (GFP), enhanced green fluorescent protein (EGFP), yellow fluorescent protein (YFP), enhanced yellow fluorescent protein (EYFP), blue fluorescent protein (BFP), red fluorescent protein (RFP), firefly luciferase, Renilla luciferase, NADPH, beta-galactosidase, horseradish peroxidase, glucose oxidase, alkaline phosphatase, chloramphenicol acetyl transferase, and urease.

In some embodiments, the one or more first oligonucleotides and second oligonucleotides bind to a different region of the target nucleic acid, or target site. In a pair, each target site is different, and the target sites are adjacent sites on the target nucleic acid, e.g. usually not more than 15 nucleotides distant, e.g. not more than 10, 8, 6, 4, or 2 nucleotides distant from the other site, and may be contiguous sites. Target sites are typically present on the same strand of the target nucleic acid in the same orientation. Target sites are also selected to provide a unique binding site, relative to other nucleic acids present in the cell. Each target site is generally from about 19 to about 25 nucleotides in length, e.g. from about 19 to 23 nucleotides, from about 19 to 21 nucleotides, or from about 19 to 20 nucleotides. The pair of first and second oligonucleotides are selected such that each oligonucleotide in the pair has a similar melting temperature for binding to its cognate target site, e.g. the Tₘ may be from about 50° C., from about 52° C., from about 55° C., from about 58°, from about 62° C., from about 65° C., from about 70° C., or from about 72° C. The GC content of the target site is generally selected to be no more than about 20%, no more than about 30%, no more than about 40%, no more than about 50%, no more than about 60%, no more than about 70%,

In some embodiments, the first oligonucleotide includes a first, second, and third complementarity region. The target site of the first oligonucleotide may refer to the first complementarity region. As summarized above, the first complementarity region of the first oligonucleotide may have a length of 19-25 nucleotides. In certain aspects, the second complementarity region of the first oligonucleotide has a length of 3-10 nucleotides, including, e.g., 4-8 nucleotides or 4-7 nucleotides. In some aspects, the second complementarity region of the first oligonucleotide has a length of 6 nucleotides. In some embodiments, the third complementarity region of the first oligonucleotide likewise has a length of 6 nucleotides. In such embodiments, the third complementarity region of the first oligonucleotide has a length of 3-10 nucleotides, including, e.g., 4-8 nucleotides or 4-7 nucleotides.

In some embodiments, second first oligonucleotide includes a first, second, and third complementarity region. The target site of the second oligonucleotide may refer to the second complementarity region. As summarized above, the second complementarity region of the second oligonucleotide may have a length of 19-25 nucleotides. In certain aspects, the first complementarity region of the first oligonucleotide has a length of 3-10 nucleotides, including, e.g., 4-8 nucleotides or 4-7 nucleotides. In some aspects, the first complementarity region of the first oligonucleotide has a length of 6 nucleotides. In some aspects, the first complementarity region of the second oligonucleotide includes the 5′ end of the second oligonucleotide. In some embodiments, the third complementarity region of the second oligonucleotide likewise has a length of 6 nucleotides. In such embodiments, the third complementarity region of the second oligonucleotide has a length of 3-10 nucleotides, including, e.g., 4-8 nucleotides or 4-7 nucleotides. In further embodiments, the third complementarity region of the second oligonucleotide includes the 3′ end of the second oligonucleotide. In some embodiments, the first complementarity region of the second oligonucleotide is adjacent to the third complementarity region of the second oligonucleotide.

In some aspects, the second oligonucleotide includes a barcode sequence, wherein the barcode sequence of the second oligonucleotide provides barcoding information for identification of the target nucleic acid. The term “barcode” refers to a nucleic acid sequence that is used to identify a single cell or a subpopulation of cells. Barcode sequences can be linked to a target nucleic acid of interest during amplification and used to trace back the amplicon to the cell from which the target nucleic acid originated. A barcode sequence can be added to a target nucleic acid of interest during amplification by carrying out amplification with an oligonucleotide that contains a region including the barcode sequence and a region that is complementary to the target nucleic acid such that the barcode sequence is incorporated into the final amplified target nucleic acid product (i.e., amplicon).

In some embodiments, the first oligonucleotide comprises a unique matching sequence (SNAIL match sequence), which is used for complementing each end of the second oligonucleotide (SNAIL shell where the ends hybridize together and are subsequently ligated). In some embodiments, the second complementarity region of the first oligonucleotide comprises a first portion of the unique matching sequence and the third complementarity region of the first oligonucleotide comprises the second portion of the unique matching sequence, and the first complementarity region of the second oligonucleotide comprises a sequence that is complementary to the first portion of the unique matching sequence, and the third complementarity region of the second oligonucleotide comprises a sequence that is complementary to the second portion of the unique matching sequence. The unique matching sequences minimize spurious probe-probe interactions that could occur either due to off-target hybridization, protein binding, or other probe aggregation or persistent close interactions, and improve specificity when greater numbers of genes are targeted or higher probe concentrations are used. The method relies on the specificity of the ligase, wherein a ligase is used that does not tolerate mismatched sequences. For detecting a plurality of different nucleic acid targets, unique matching sequences may be generated per gene or per probe pair.

In some embodiments, the first oligonucleotide further comprises a common binding site for a gel adaptor oligonucleotide. The gel adaptor oligonucleotide comprises a functional attachment modification at its 5′ end or 3′ end, such as acrydite, such that the first oligonucleotide is covalently linked via the gel adaptor oligonucleotide to the hydrogel during gelation. The use of a gel adaptor helps to retain amplicons grown from the 3′ end of the first oligonucleotide in a gel, without the need for the first oligonucleotide to have a 5′ modification itself. In some embodiments, the common binding site for the gel adaptor oligonucleotide is adjacent to the first complementarity region of the first oligonucleotide.

In certain embodiments, an mRNA retention oligonucleotide is used to retain mRNA in the hydrogel during or prior to hybridization. The mRNA retention oligonucleotide comprises: a nucleotide modification at the 5′ end or 3′ end such as an acrydite modification, wherein the mRNA retention oligonucleotide is linked to the hydrogel during gelation; a poly-T tail that hybridizes to a poly-A tail of an mRNA, wherein hybridization of the poly-T tail of the mRNA retention oligonucleotide to the poly-A tail of the mRNA retains the mRNA in the hydrogel; and a unique hybridization sequence. In some embodiments, the poly-T tail comprises interleaved locked nucleic acid (LNA) thymine (T) bases. The use of the mRNA retention oligonucleotide enables gelation to be performed before hybridization if desired. The unique hybridization sequence on the mRNA retention oligonucleotide allows labeling of mRNA by hybridization with a complementary fluorescently labeled oligonucleotide probe.

### Tissue

As described herein, the methods disclosed include in situ sequencing technology of an intact tissue by at least contacting a fixed and permeabilized intact tissue with at least a pair of oligonucleotide primers under conditions to allow for specific hybridization. Tissue specimens suitable for use with the methods described herein generally include any type of tissue specimens collected from living or dead subjects, such as, e.g., biopsy specimens and autopsy specimens, of which include, but are not limited to, epithelium, muscle, connective, and nervous tissue. Tissue specimens may be collected and processed using the methods described herein and subjected to microscopic analysis immediately following processing, or may be preserved and subjected to microscopic analysis at a future time, e.g., after storage for an extended period of time. In some embodiments, the methods described herein may be used to preserve tissue specimens in a stable, accessible and fully intact form for future analysis. In some embodiments, the methods described herein may be used to analyze a previously-preserved or stored tissue specimen. In some embodiments, the intact tissue includes brain tissue such as visual cortex slices. In some embodiments, the intact tissue is a thin slice with a thickness of 5-20 μm, including, but not limited to, e.g., 5-18 μm, 5-15 μm, or 5-10 μm. In other embodiments, the intact tissue is a thick slice with a thickness of 50-200 μm, including, but not limited to, e.g., 50-150 μm, 50-100 μm, or 50-80 μm.

Aspects of the invention include fixing intact tissue. The term “fixing” or “fixation” as used herein is the process of preserving biological material (e.g., tissues, cells, organelles, molecules, etc.) from decay and/or degradation. Fixation may be accomplished using any convenient protocol. Fixation can include contacting the sample with a fixation reagent (i.e., a reagent that contains at least one fixative). Samples can be contacted by a fixation reagent for a wide range of times, which can depend on the temperature, the nature of the sample, and on the fixative(s). For example, a sample can be contacted by a fixation reagent for 24 or less hours, 18 or less hours, 12 or less hours, 8 or less hours, 6 or less hours, 4 or less hours, 2 or less hours, 60 or less minutes, 45 or less minutes, 30 or less minutes, 25 or less minutes, 20 or less minutes, 15 or less minutes, 10 or less minutes, 5 or less minutes, or 2 or less minutes.

A sample can be contacted by a fixation reagent for a period of time in a range of from 5 minutes to 24 hours, e.g., from 10 minutes to 20 hours, from 10 minutes to 18 hours, from 10 minutes to 12 hours, from 10 minutes to 8 hours, from 10 minutes to 6 hours, from 10 minutes to 4 hours, from 10 minutes to 2 hours, from 15 minutes to 20 hours, from 15 minutes to 18 hours, from 15 minutes to 12 hours, from 15 minutes to 8 hours, from 15 minutes to 6 hours, from 15 minutes to 4 hours, from 15 minutes to 2 hours, from 15 minutes to 1.5 hours, from 15 minutes to 1 hour, from 10 minutes to 30 minutes, from 15 minutes to 30 minutes, from 30 minutes to 2 hours, from 45 minutes to 1.5 hours, or from 55 minutes to 70 minutes.

A sample can be contacted by a fixation reagent at various temperatures, depending on the protocol and the reagent used. For example, in some instances a sample can be contacted by a fixation reagent at a temperature ranging from −22° C. to 55° C., where specific ranges of interest include, but are not limited to 50 to 54° C., 40 to 44° C., 35 to 39° C., 28 to 32° C., 20 to 26° C., 0 to 6° C., and −18 to −22° C. In some instances a sample can be contacted by a fixation reagent at a temperature of −20° C., 4° C., room temperature (22-25° C.), 30° C., 37° C., 42° C., or 52° C.

Any convenient fixation reagent can be used. Common fixation reagents include crosslinking fixatives, precipitating fixatives, oxidizing fixatives, mercurials, and the like. Crosslinking fixatives chemically join two or more molecules by a covalent bond and a wide range of cross-linking reagents can be used. Examples of suitable cross-liking fixatives include but are not limited to aldehydes (e.g., formaldehyde, also commonly referred to as “paraformaldehyde” and “formalin”; glutaraldehyde; etc.), imidoesters, NHS (N-Hydroxysuccinimide) esters, and the like. Examples of suitable precipitating fixatives include but are not limited to alcohols (e.g., methanol, ethanol, etc.), acetone, acetic acid, etc. In some embodiments, the fixative is formaldehyde (i.e., paraformaldehyde or formalin). A suitable final concentration of formaldehyde in a fixation reagent is 0.1 to 10%, 1-8%, 1-4%, 1-2%, 3-5%, or 3.5-4.5%, including about 1.6% for 10 minutes. In some embodiments the sample is fixed in a final concentration of 4% formaldehyde (as diluted from a more concentrated stock solution, e.g., 38%, 37%, 36%, 20%, 18%, 16%, 14%, 10%, 8%, 6%, etc.). In some embodiments the sample is fixed in a final concentration of 10% formaldehyde. In some embodiments the sample is fixed in a final concentration of 1% formaldehyde. In some embodiments, the fixative is glutaraldehyde. A suitable concentration of glutaraldehyde in a fixation reagent is 0.1 to 1%. A fixation reagent can contain more than one fixative in any combination. For example, in some embodiments the sample is contacted with a fixation reagent containing both formaldehyde and glutaraldehyde.

The terms “permeabilization” or “permeabilize” as used herein refer to the process of rendering the cells (cell membranes etc.) of a sample permeable to experimental reagents such as nucleic acid probes, antibodies, chemical substrates, etc. Any convenient method and/or reagent for permeabilization can be used. Suitable permeabilization reagents include detergents (e.g., Saponin, Triton X-100, Tween-20, etc.), organic fixatives (e.g., acetone, methanol, ethanol, etc.), enzymes, etc. Detergents can be used at a range of concentrations. For example, 0.001%-1% detergent, 0.05%-0.5% detergent, or 0.1%-0.3% detergent can be used for permeabilization (e.g., 0.1% Saponin, 0.2% tween-20, 0.1-0.3% triton X-100, etc.). In some embodiments methanol on ice for at least 10 minutes is used to permeabilize.

In some embodiments, the same solution can be used as the fixation reagent and the permeabilization reagent. For example, in some embodiments, the fixation reagent contains 0.1%-10% formaldehyde and 0.001%-1% saponin. In some embodiments, the fixation reagent contains 1% formaldehyde and 0.3% saponin.

A sample can be contacted by a permeabilization reagent for a wide range of times, which can depend on the temperature, the nature of the sample, and on the permeabilization reagent(s). For example, a sample can be contacted by a permeabilization reagent for 24 or more hours, 24 or less hours, 18 or less hours, 12 or less hours, 8 or less hours, 6 or less hours, 4 or less hours, 2 or less hours, 60 or less minutes, 45 or less minutes, 30 or less minutes, 25 or less minutes, 20 or less minutes, 15 or less minutes, 10 or less minutes, 5 or less minutes, or 2 or less minutes. A sample can be contacted by a permeabilization reagent at various temperatures, depending on the protocol and the reagent used. For example, in some instances a sample can be contacted by a permeabilization reagent at a temperature ranging from −82° C. to 55° C., where specific ranges of interest include, but are not limited to: 50 to 54° C., 40 to 44° C., 35 to 39° C., 28 to 32° C., 20 to 26° C., 0 to 6° C., −18 to −22° C., and −78 to −82° C. In some instances a sample can be contacted by a permeabilization reagent at a temperature of −80° C., −20° C., 4° C., room temperature (22-25° C.), 30° C., 37° C., 42° C., or 52° C.

In some embodiments, a sample is contacted with an enzymatic permeabilization reagent. Enzymatic permeabilization reagents that permeabilize a sample by partially degrading extracellular matrix or surface proteins that hinder the permeation of the sample by assay reagents. Contact with an enzymatic permeabilization reagent can take place at any point after fixation and prior to target detection. In some instances the enzymatic permeabilization reagent is proteinase K, a commercially available enzyme. In such cases, the sample is contacted with proteinase K prior to contact with a post-fixation reagent. Proteinase K treatment (i.e., contact by proteinase K; also commonly referred to as “proteinase K digestion”) can be performed over a range of times at a range of temperatures, over a range of enzyme concentrations that are empirically determined for each cell type or tissue type under investigation. For example, a sample can be contacted by proteinase K for 30 or less minutes, 25 or less minutes, 20 or less minutes, 15 or less minutes, 10 or less minutes, 5 or less minutes, or 2 or less minutes. A sample can be contacted by 1 μg/ml or less, 2 μg/m or less, 4 μg/ml or less, 8 μg/ml or less, 10 μg/ml or less, 20 μg/ml or less, 30 μg/ml or less, 50 μg/ml or less, or 100 μg/ml or less proteinase K. A sample can be contacted by proteinase K at a temperature ranging from 2° C. to 55° C., where specific ranges of interest include, but are not limited to: 50 to 54° C., 40 to 44° C., 35 to 39° C., 28 to 32° C., 20 to 26° C., and 0 to 6° C. In some instances a sample can be contacted by proteinase K at a temperature of 4° C., room temperature (22-25° C.), 30° C., 37° C., 42° C., or 52° C. In some embodiments, a sample is not contacted with an enzymatic permeabilization reagent. In some embodiments, a sample is not contacted with proteinase K. Contact of an intact tissue with at least a fixation reagent and a permeabilization reagent results in the production of a fixed and permeabilized tissue.

### Ligase

In some embodiments, the methods disclosed include adding ligase to ligate the second oligonucleotide and generate a closed nucleic acid circle. In some embodiments, the adding ligase includes adding DNA ligase. In alternative embodiments, the second oligonucleotide is provided as a closed nucleic acid circle, and the step of adding ligase is omitted. In certain embodiments, ligase is an enzyme that facilitates the sequencing of a target nucleic acid molecule.

The term “ligase” as used herein refers to an enzyme that is commonly used to join polynucleotides together or to join the ends of a single polynucleotide. Ligases include ATP-dependent double-strand polynucleotide ligases, NAD-i-dependent double-strand DNA or RNA ligases and single-strand polynucleotide ligases, for example any of the ligases described in EC 6.5.1.1 (ATP-dependent ligases), EC 6.5.1.2 (NAD+-dependent ligases), EC 6.5.1.3 (RNA ligases). Specific examples of ligases include bacterial ligases such as E. coli DNA ligase and Taq DNA ligase, Ampligase® thermostable DNA ligase (Epicentre®Technologies Corp., part of Illumina®, Madison, Wis.) and phage ligases such as T3 DNA ligase, T4 DNA ligase and T7 DNA ligase and mutants thereof.

### Rolling Circle Amplification

In some embodiments, the methods of the invention include the step of performing rolling circle amplification in the presence of a nucleic acid molecule, wherein the performing includes using the second oligonucleotide as a template and the first oligonucleotide as a primer for a polymerase to form one or more amplicons. In such embodiments, a single-stranded, circular polynucleotide template is formed by ligation of the second nucleotide, which circular polynucleotide includes a region that is complementary to the first oligonucleotide. Upon addition of a DNA polymerase in the presence of appropriate dNTP precursors and other cofactors, the first oligonucleotide is elongated by replication of multiple copies of the template. This amplification product can be readily detected by binding to a detection probe. In some embodiments, the polymerase is preincubated without dNTPs to allow the polymerase to penetrate the sample uniformly before performing rolling circle amplification.

In some embodiments, only when a first oligonucleotide and second oligonucleotide hybridize to the same target nucleic acid molecule, the second oligonucleotide can be circularized and rolling-circle amplified to generate a cDNA nanoball (i.e., amplicon) containing multiple copies of the cDNA. The term “amplicon” refers to the amplified nucleic acid product of a PCR reaction or other nucleic acid amplification process. In some embodiments, amine-modified nucleotides are spiked into the rolling circle amplification reaction.

Techniques for rolling circle amplification are known in the art (see, e.g., Baner et al, Nucleic Acids Research, 26:5073-5078, 1998; Lizardi et al, Nature Genetics 19:226, 1998; Schweitzer et al. Proc. Natl Acad. Sci. USA 97:101 13-1 19, 2000; Faruqi et al, BMC Genomics 2:4, 2000; Nallur et al, Nucl. Acids Res. 29:el 18, 2001; Dean et al. Genome Res. 1 1:1095-1099, 2001; Schweitzer et al, Nature Biotech. 20:359-365, 2002; U.S. Pat. Nos. 6,054,274, 6,291,187, 6,323,009, 6,344,329 and 6,368,801). In some embodiments the polymerase is phi29 DNA polymerase.

In certain aspects, the nucleic acid molecule includes an amine-modified nucleotide. In such embodiments, the amine-modified nucleotide includes an acrylic acid N-hydroxysuccinimide moiety modification. Examples of other amine-modified nucleotides include, but are not limited to, a 5-Aminoallyl-dUTP moiety modification, a 5-Propargylamino-dCTP moiety modification, a N6-6-Aminohexyl-dATP moiety modification, or a 7-Deaza-7-Propargylamino-dATP moiety modification.

In some embodiments, rolling circle amplification is performed in the presence of an oligonucleotide that labels and condenses amplicons (“amplicon condensing and detection oligonucleotide”). The amplicon condensing and detection oligonucleotide comprises an end modification for attachment to the gel, such as a 5′ primary amine or acrydite modification. In addition, the amplicon condensing and detection oligonucleotide comprises a unique sequence for hybridization of a probe for amplicon detection and two or more copies of a sequence complementary to a common sequence on the amplicons such that the amplicon condensing and detection oligonucleotide draws together strands from a given amplicon. In some embodiments, the amplicon condensing and detection oligonucleotide comprises three or more, four or more, five or more, six or more, seven or more, eight or more, nine or more, or ten or more copies of the sequence complementary to the common sequence on the amplicons. The use of amplicon condensing and detection oligonucleotides decreases the optical spread of an amplicon and makes it less likely that adjacent amplicons from different genes aggregate when densely packed. In some embodiments, an amplicon condensing and detection oligonucleotide is used for in situ gene sequencing of target nucleic acids in a thick tissue section.

### Amplicon Embedding in a Tissue-Hydrogel Setting

In some embodiments, the methods disclosed include embedding one or more amplicons in the presence of hydrogel subunits to form one or more hydrogel-embedded amplicons. The hydrogel-tissue chemistry described includes covalently attaching nucleic acids to in situ synthesized hydrogel for tissue clearing, enzyme diffusion, and multiple-cycle sequencing while an existing hydrogel-tissue chemistry method cannot. In some embodiments, to enable amplicon embedding in the tissue-hydrogel setting, amine-modified nucleotides are spiked into the rolling circle amplification reaction, functionalized with an acrylamide moiety using acrylic acid N-hydroxysuccinimide esters, and copolymerized with acrylamide monomers to form a hydrogel.

As used herein, the terms “hydrogel” or “hydrogel network” mean a network of polymer chains that are water-insoluble, sometimes found as a colloidal gel in which water is the dispersion medium. In other words, hydrogels are a class of polymeric materials that can absorb large amounts of water without dissolving. Hydrogels can contain over 99% water and may include natural or synthetic polymers, or a combination thereof. Hydrogels also possess a degree of flexibility very similar to natural tissue, due to their significant water content. A detailed description of suitable hydrogels may be found in published U.S. patent application 20100055733, herein specifically incorporated by reference. As used herein, the terms “hydrogel subunits” or “hydrogel precursors” mean hydrophilic monomers, prepolymers, or polymers that can be crosslinked, or “polymerized”, to form a three-dimensional (3D) hydrogel network. Without being bound by any scientific theory, it is believed that this fixation of the biological specimen in the presence of hydrogel subunits crosslinks the components of the specimen to the hydrogel subunits, thereby securing molecular components in place, preserving the tissue architecture and cell morphology.

In some embodiments, the embedding includes copolymerizing the one or more amplicons with acrylamide. As used herein, the term “copolymer” describes a polymer which contains more than one type of subunit. The term encompasses polymer which include two, three, four, five, or six types of subunits.

In certain aspects, the embedding includes clearing the one or more hydrogel-embedded amplicons wherein the target nucleic acid is substantially retained in the one or more hydrogel-embedded amplicons. In such embodiments, the clearing includes substantially removing a plurality of cellular components from the one or more hydrogel-embedded amplicons. In some other embodiments, the clearing includes substantially removing lipids and/or proteins from the one or more hydrogel-embedded amplicons. As used herein, the term “substantially” means that the original amount present in the sample before clearing has been reduced by approximately 70% or more, such as by 75% or more, such as by 80% or more, such as by 85% or more, such as by 90% or more, such as by 95% or more, such as by 99% or more, such as by 100%.

In some embodiments, clearing the hydrogel-embedded amplicons includes performing electrophoresis on the specimen. In some embodiments, the amplicons are electrophoresed using a buffer solution that includes an ionic surfactant. In some embodiments, the ionic surfactant is sodium dodecyl sulfate (SDS). In some embodiments, the specimen is electrophoresed using a voltage ranging from about 10 to about 60 volts. In some embodiments, the specimen is electrophoresed for a period of time ranging from about 15 minutes up to about 10 days. In some embodiments, the methods further involve incubating the cleared specimen in a mounting medium that has a refractive index that matches that of the cleared tissue. In some embodiments, the mounting medium increases the optical clarity of the specimen. In some embodiments, the mounting medium includes glycerol.

### SCAL and SEDAL2 Sequencing-by-Ligation

In some embodiments, SEDAL2 or SCAL sequencing-by-ligation methods are used. The methods disclosed herein include the step of contacting one or more hydrogel-embedded amplicons having a barcode sequence with a pair of primers under conditions to allow for ligation, wherein the pair of primers include a third oligonucleotide and a fourth oligonucleotide, wherein ligation only occurs when both the third oligonucleotide and the fourth oligonucleotide ligate to the same amplicon. In some embodiments, the third oligonucleotide is configured to decode bases and the fourth oligonucleotide is configured to convert decoded bases into a signal. In some aspects, the signal is a fluorescent signal. In exemplary aspects, the contacting the one or more hydrogel-embedded amplicons having the barcode sequence with a pair of primers under conditions to allow for ligation involves each of the third oligonucleotide and the fourth oligonucleotide ligating to form a stable product for imaging only when a perfect match occurs. In certain aspects, the mismatch sensitivity of a ligase enzyme is used to determine the underlying sequence of the target nucleic acid molecule.

Inclusion of a polyethylene glycol (PEG) polymer in the sequencing ligation mixture substantially accelerates signal addition onto target nucleic acids. Exemplary PEG polymers have molecular weights ranging from 300 g/mol to 10,000,000 g/mol. In some embodiments, a PEG 6000 polymer is present during ligation of the third and fourth oligonucleotides.

In some embodiments, the contacting the one or more hydrogel-embedded amplicons occurs two times or more, including, but not limited to, e.g., three times or more, four times or more, five times or more, six times or more, or seven times or more. In certain embodiments, the contacting the one or more hydrogel-embedded amplicons occurs four times or more for thin tissue specimens. In other embodiments, the contacting the one or more hydrogel-embedded amplicons occurs six times or more for thick tissue specimens. In some embodiments, one or more amplicons can be contacted by a pair of primers for 24 or more hours, 24 or less hours, 18 or less hours, 12 or less hours, 8 or less hours, 6 or less hours, 4 or less hours, 2 or less hours, 60 or less minutes, 45 or less minutes, 30 or less minutes, 25 or less minutes, 20 or less minutes, 15 or less minutes, 10 or less minutes, 5 or less minutes, or 2 or less minutes. In some embodiments, the methods are performed at room temperature for preservation of tissue morphology with low background noise and error reduction. In some embodiments, the contacting the one or more hydrogel-embedded amplicons includes eliminating error accumulation as sequencing proceeds.

Specimens prepared using the subject methods may be analyzed by any of a number of different types of microscopy, for example, optical microscopy (e.g. bright field, oblique illumination, dark field, phase contrast, differential interference contrast, interference reflection, epifluorescence, confocal, etc., microscopy), laser microscopy, electron microscopy, and scanning probe microscopy. In some aspects, a non-transitory computer readable medium transforms raw images acquired through microscopy of multiple rounds of in situ sequencing first into decoded gene identities and spatial locations and then analyzes the per-cell composition of gene expression.

The term “perfectly matched”, when used in reference to a duplex means that the polynucleotide and/or oligonucleotide strands making up the duplex form a double stranded structure with one another such that every nucleotide in each strand undergoes Watson-Crick base pairing with a nucleotide in the other strand. The term “duplex” includes, but is not limited to, the pairing of nucleoside analogs, such as deoxyinosine, nucleosides with 2-aminopurine bases, peptide nucleic acids (PNAs), and the like, that may be employed. A “mismatch” in a duplex between two oligonucleotides means that a pair of nucleotides in the duplex fails to undergo Watson-Crick bonding.

In some embodiments, the method includes a plurality of third oligonucleotides, including, but not limited to, 5 or more third oligonucleotides, e.g., 8 or more, 10 or more, 12 or more, 15 or more, 18 or more, 20 or more, 25 or more, 30 or more, 35 or more that hybridize to target nucleotide sequences. In some embodiments, a method of the present disclosure includes a plurality of third oligonucleotides, including, but not limited to, 15 or more third oligonucleotides, e.g., 20 or more, 30 or more, 40 or more, 50 or more, 60 or more, 70 or more, and up to 80 different first oligonucleotides that hybridize to 15 or more, e.g., 20 or more, 30 or more, 40 or more, 50 or more, 60 or more, 70 or more, and up to 80 different target nucleotide sequences. In some embodiments, the methods include a plurality of fourth oligonucleotides, including, but not limited to, 5 or more fourth oligonucleotides, e.g., 8 or more, 10 or more, 12 or more, 15 or more, 18 or more, 20 or more, 25 or more, 30 or more, 35 or more. In some embodiments, a method of the present disclosure includes a plurality of fourth oligonucleotides including, but not limited to, 15 or more fourth oligonucleotides, e.g., 20 or more, 30 or more, 40 or more, 50 or more, 60 or more, 70 or more, and up to 80 different first oligonucleotides that hybridize to 15 or more, e.g., 20 or more, 30 or more, 40 or more, 50 or more, 60 or more, 70 or more, and up to 80 different target nucleotide sequences. A plurality of oligonucleotide pairs can be used in a reaction, where one or more pairs specifically bind to each target nucleic acid. For example, two primer pairs can be used for one target nucleic acid in order to improve sensitivity and reduce variability. It is also of interest to detect a plurality of different target nucleic acids in a cell, e.g. detecting up to 2, up to 3, up to 4, up to 5, up to 6, up to 7, up to 8, up to 9, up to 10, up to 12, up to 15, up to 18, up to 20, up to 25, up to 30, up to 40 or more distinct target nucleic acids.

In certain embodiments, sequencing is performed with a ligase with activity hindered by base mismatches, a third oligonucleotide, and a fourth oligonucleotide. The term “hindered” in this context refers to activity of a ligase that is reduced by approximately 20% or more, such as by 25% or more, such as by 50% or more, such as by 75% or more, such as by 90% or more, such as by 95% or more, such as by 99% or more, such as by 100%. In some embodiments, the third oligonucleotide has a length of 5-15 nucleotides, including, but not limited to, 5-13 nucleotides, 5-10 nucleotides, or 5-8 nucleotides. In some embodiments, the Tₘ of the third oligonucleotide is at room temperature (22-25° C.). In some embodiments, the third oligonucleotide is degenerate, or partially thereof. In some embodiments, the fourth oligonucleotide has a length of 5-15 nucleotides, including, but not limited to, 5-13 nucleotides, 5-10 nucleotides, or 5-8 nucleotides. In some embodiments, the Tₘ of the fourth oligonucleotide is at room temperature (22°−25° C.). After each cycle of sequencing corresponding to a base readout, the fourth oligonucleotides may be stripped, which eliminates error accumulation as sequencing proceeds. In some embodiments, the fourth oligonucleotides are stripped by formamide.

In some embodiments, sequencing involves the washing of the third oligonucleotide and the fourth oligonucleotide to remove unbound oligonucleotides, thereafter revealing a fluorescent product for imaging. In certain exemplary embodiments, a detectable label can be used to detect one or more nucleotides and/or oligonucleotides described herein. In certain embodiments, a detectable label can be used to detect the one or more amplicons. Examples of detectable markers include various radioactive moieties, enzymes, prosthetic groups, fluorescent markers, luminescent markers, bioluminescent markers, metal particles, protein-protein binding pairs, protein-antibody binding pairs and the like. Examples of fluorescent proteins include, but are not limited to, yellow fluorescent protein (YFP), green fluorescence protein (GFP), cyan fluorescence protein (CFP), umbelliferone, fluorescein, fluorescein isothiocyanate, rhodamine, dichlorotriazinylamine fluorescein, dansyl chloride, phycoerythrin and the like. Examples of bioluminescent markers include, but are not limited to, luciferase (e.g., bacterial, firefly, click beetle and the like), luciferin, aequorin and the like. Examples of enzyme systems having visually detectable signals include, but are not limited to, galactosidases, glucorimidases, phosphatases, peroxidases, cholinesterases and the like. Identifiable markers also include radioactive compounds such as ¹²⁵I, ³⁵S, ¹⁴C, or ³H. Identifiable markers are commercially available from a variety of sources.

Fluorescent labels and their attachment to nucleotides and/or oligonucleotides are described in many reviews, including Haugland, Handbook of Fluorescent Probes and Research Chemicals, Ninth Edition (Molecular Probes, Inc., Eugene, 2002); Keller and Manak, DNA Probes, 2nd Edition (Stockton Press, New York, 1993); Eckstein, editor, Oligonucleotides and Analogues: A Practical Approach (IRL Press, Oxford, 1991); and Wetmur, Critical Reviews in Biochemistry and Molecular Biology, 26:227-259 (1991). Particular methodologies applicable to the invention are disclosed in the following sample of references: U.S. Pat. Nos. 4,757,141, 5,151,507 and 5,091,519. In one aspect, one or more fluorescent dyes are used as labels for labeled target sequences, e.g., as disclosed by U.S. Pat. No. 5,188,934 (4,7-dichlorofluorescein dyes); U.S. Pat. No. 5,366,860 (spectrally resolvable rhodamine dyes); U.S. Pat. No. 5,847,162 (4,7-dichlororhodamine dyes); U.S. Pat. No. 4,318,846 (ether-substituted fluorescein dyes); U.S. Pat. No. 5,800,996 (energy transfer dyes); Lee et al.; U.S. Pat. No. 5,066,580 (xanthine dyes); U.S. Pat. No. 5,688,648 (energy transfer dyes); and the like. Labelling can also be carried out with quantum dots, as disclosed in the following patents and patent publications: U.S. Pat. Nos. 6,322,901, 6,576,291, 6,423,551, 6,251,303, 6,319,426, 6,426,513, 6,444,143, 5,990,479, 6,207,392, 2002/0045045 and 2003/0017264. As used herein, the term “fluorescent label” includes a signaling moiety that conveys information through the fluorescent absorption and/or emission properties of one or more molecules. Such fluorescent properties include fluorescence intensity, fluorescence lifetime, emission spectrum characteristics, energy transfer, and the like.

Commercially available fluorescent nucleotide analogues readily incorporated into nucleotide and/or oligonucleotide sequences include, but are not limited to, Cy3-dCTP, Cy3-dUTP, Cy5-dCTP, Cy5-dUTP (Amersham Biosciences, Piscataway, N.J.), fluorescein-12-dUTP, tetramethylrhodamine-6-dUTP, TEXAS RED™-5-dUTP, CASCADE BLUE™-7-dUTP, BODIPY TMFL-14-dUTP, BODIPY TMR-14-dUTP, BODIPY TMTR-14-dUTP, RHODAMINE GREEN™-5-dUTP, OREGON GREENR™ 488-5-dUTP, TEXAS RED™-12-dUTP, BODIPY™ 630/650-14-dUTP, BODIPY™ 650/665-14-dUTP, ALEXA FLUOR™ 488-5-dUTP, ALEXA FLUOR™ 532-5-dUTP, ALEXA FLUOR™ 568-5-dUTP, ALEXA FLUOR™ 594-5-dUTP, ALEXA FLUOR™ 546-14-dUTP, fluorescein-12-UTP, tetramethylrhodamine-6-UTP, TEXAS RED™-5-UTP, mCherry, CASCADE BLUE™-7-UTP, BODIPY™ FL-14-UTP, BODIPY TMR-14-UTP, BODIPY™ TR-14-UTP, RHODAMINE GREEN™-5-UTP, ALEXA FLUOR™ 488-5-UTP, LEXA FLUOR™ 546-14-UTP (Molecular Probes, Inc. Eugene, Oreg.) and the like. Protocols are known in the art for custom synthesis of nucleotides having other fluorophores (See, Henegariu et al. (2000) Nature Biotechnol. 18:345).

Other fluorophores available for post-synthetic attachment include, but are not limited to, ALEXA FLUOR™ 350, ALEXA FLUOR™ 532, ALEXA FLUOR™ 546, ALEXA FLUOR™ 568, ALEXA FLUOR™ 594, ALEXA FLUOR™ 647, BODIPY 493/503, BODIPY FL, BODIPY R6G, BODIPY 530/550, BODIPY TMR, BODIPY 558/568, BODIPY 558/568, BODIPY 564/570, BODIPY 576/589, BODIPY 581/591, BODIPY 630/650, BODIPY 650/665, Cascade Blue, Cascade Yellow, Dansyl, lissamine rhodamine B, Marina Blue, Oregon Green 488, Oregon Green 514, Pacific Blue, rhodamine 6G, rhodamine green, rhodamine red, tetramethyl rhodamine, Texas Red (available from Molecular Probes, Inc., Eugene, Oreg.), Cy2, Cy3.5, Cy5.5, Cy7 (Amersham Biosciences, Piscataway, N.J.) and the like. FRET tandem fluorophores may also be used, including, but not limited to, PerCP-Cy5.5, PE-Cy5, PE-Cy5.5, PE-Cy7, PE-Texas Red, APC-Cy7, PE-Alexa dyes (610, 647, 680), APC-Alexa dyes and the like.

Metallic silver or gold particles may be used to enhance signal from fluorescently labeled nucleotide and/or oligonucleotide sequences (Lakowicz et al. (2003) Bio Techniques 34:62).

Biotin, or a derivative thereof, may also be used as a label on a nucleotide and/or an oligonucleotide sequence, and subsequently bound by a detectably labeled avidin/streptavidin derivative (e.g. phycoerythrin-conjugated streptavidin), or a detectably labeled anti-biotin antibody. Digoxigenin may be incorporated as a label and subsequently bound by a detectably labeled anti-digoxigenin antibody (e.g. fluoresceinated anti-digoxigenin). An aminoallyl-dUTP residue may be incorporated into an oligonucleotide sequence and subsequently coupled to an N-hydroxy succinimide (NHS) derivatized fluorescent dye. In general, any member of a conjugate pair may be incorporated into a detection oligonucleotide provided that a detectably labeled conjugate partner can be bound to permit detection. As used herein, the term antibody refers to an antibody molecule of any class, or any sub-fragment thereof, such as an Fab.

Other suitable labels for an oligonucleotide sequence may include fluorescein (FAM), digoxigenin, dinitrophenol (DNP), dansyl, biotin, bromodeoxyuridine (BrdU), hexahistidine (6×His), phosphor-amino acids (e.g. P-tyr, P-ser, P-thr) and the like. In one embodiment the following hapten/antibody pairs are used for detection, in which each of the antibodies is derivatized with a detectable label: biotin/α-biotin, digoxigenin/α-digoxigenin, dinitrophenol (DNP)/α-DNP, 5-Carboxyfluorescein (FAM)/α-FAM.

In certain exemplary embodiments, a nucleotide and/or an oligonucleotide sequence can be indirectly labeled, especially with a hapten that is then bound by a capture agent, e.g., as disclosed in U.S. Pat. Nos. 5,344,757, 5,702,888, 5,354,657, 5,198,537 and 4,849,336, PCT publication WO 91/17160 and the like. Many different hapten-capture agent pairs are available for use. Exemplary haptens include, but are not limited to, biotin, des-biotin and other derivatives, dinitrophenol, dansyl, fluorescein, CY5, digoxigenin and the like. For biotin, a capture agent may be avidin, streptavidin, or antibodies. Antibodies may be used as capture agents for the other haptens (many dye-antibody pairs being commercially available, e.g., Molecular Probes, Eugene, Oreg.).

In some embodiments, an antioxidant compound is included in the washing and imaging buffers (i.e., “anti-fade buffers”) to reduce photobleaching during fluorescence imaging. Exemplary antioxidants include, without limitation, trolox (6-hydroxy-2,5,7,8-tetramethylchroman-2-carboxylic acid) and trolox-quinone, propyl-gallate, tertiary butylhydroquinone, butylated hydroxyanisole, butylated hydroxytoluene, glutathione, ascorbic acid, and tocopherols. Such antioxidants have an antifade effect on fluorophores. That is, the antioxidant reduces photobleaching during tiling, greatly enhances the signal-to-noise ratio (SNR) of sensitive fluorophores, and enables higher SNR imaging of thicker samples. For a fixed exposure time, including an antioxidant increases the SNR by increasing the concentration of the non-bleached fluorophore during exposure to light. Including an antioxidant also removes the diminishing returns of longer exposure times (caused by the limited fluorophore lifetime before photobleaching), providing for increased SNR by allowing increased exposure times.

In addition, fluorophore cleavage from probes or probe stripping can be used to eliminate signal carryover from one round to the next when multiple sequencing cycles are used. For example, fluorophores can be stripped off with formamide. Alternatively, thiol-linked dyes can be used having a disulfide linkage between the fluorophore and an oligonucleotide probe, which enables cleavage of the fluorophore from the oligonucleotide probe in a reducing environment. Exemplary disulfide reducing agents, which can be used for cleaving disulfide bonds include, without limitation, tris(2-carboxyethyl)phosphine (TCEP), dithiothreitol (DTT), and β-mercaptoethanol (BME). Following fluorescence imaging during a sequencing round, a stripping agent and/or a reducing agent is added, and subsequent washing steps remove the diffusive fluorescent signal before performing another round of sequencing.

A sequencing cycle for SCAL or SEDAL2 optionally begins with a brief sample wash, before proceeding to the first signal addition. For SCAL sequencing, depending on whether sequential or combinatorial encoding is being used for a particular round, the corresponding set of third and fourth oligonucleotides and their round-specific competitors are added and ligated. In combinatorial encodings, the third oligonucleotide for a given position x is added, plus a set of fluorescently labeled dibase-encoding oligonucleotides, plus a competitor oligonucleotide for the previous position that was labeled (unless it is the first round of labeling, in which case competitor oligonucleotide is omitted). In sequential encodings, the third oligonucleotide for a given round x, a 4-channel fluorophore mixture, and a round x-1 competitor oligonucleotide are added, except if it is the first round of labeling. The presence of PEG in the sequencing ligation mixture substantially accelerates the signal addition onto the target. Following incubation of the sample in imaging buffer, the sample is imaged, and briefly rinsed before proceeding to the next sequencing cycle.

For SEDAL2, the same oligonucleotide/ligation mixture is used as described above during the signal addition phase, except competitor oligonucleotides are omitted. Following sample addition, washing, imaging buffer addition, and imaging as described above, SEDAL2 includes a separate phase for signal removal, in which signals are either stripped off with a formamide-containing stripping solution or if thiol-linked dyes are used for sequential encoding fluorescently labeled oligonucleotides, a cleaving solution containing a disulfide reducing agent (e.g., TCEP). Samples are subsequently washed before proceeding to the next round of signal addition.

### Cells

Methods disclosed herein include a method for in situ gene sequencing of a target nucleic acid in a cell in an intact tissue. In certain embodiments, the cell is present in a population of cells. In certain other embodiments, the population of cells includes a plurality of cell types including, but not limited to, excitatory neurons, inhibitory neurons, and non-neuronal cells. Cells for use in the assays of the invention can be an organism, a single cell type derived from an organism, or can be a mixture of cell types. Included are naturally occurring cells and cell populations, genetically engineered cell lines, cells derived from transgenic animals, etc. Virtually any cell type and size can be accommodated. Suitable cells include bacterial, fungal, plant and animal cells. In one embodiment of the invention, the cells are mammalian cells, e.g. complex cell populations such as naturally occurring tissues, for example blood, liver, pancreas, neural tissue, bone marrow, skin, and the like. Some tissues may be disrupted into a monodisperse suspension. Alternatively, the cells may be a cultured population, e.g. a culture derived from a complex population, a culture derived from a single cell type where the cells have differentiated into multiple lineages, or where the cells are responding differentially to stimulus, and the like.

Cell types that can find use in the subject invention include stem and progenitor cells, e.g. embryonic stem cells, hematopoietic stem cells, mesenchymal stem cells, neural crest cells, etc., endothelial cells, muscle cells, myocardial, smooth and skeletal muscle cells, mesenchymal cells, epithelial cells; hematopoietic cells, such as lymphocytes, including T-cells, such as Th1 T cells, Th2 T cells, ThO T cells, cytotoxic T cells; B cells, pre-B cells, etc.; monocytes; dendritic cells; neutrophils; and macrophages; natural killer cells; mast cells, etc.; adipocytes, cells involved with particular organs, such as thymus, endocrine glands, pancreas, brain, such as neurons, glia, astrocytes, dendrocytes, etc. and genetically modified cells thereof. Hematopoietic cells may be associated with inflammatory processes, autoimmune diseases, etc., endothelial cells, smooth muscle cells, myocardial cells, etc. may be associated with cardiovascular diseases; almost any type of cell may be associated with neoplasias, such as sarcomas, carcinomas and lymphomas; liver diseases with hepatic cells; kidney diseases with kidney cells; etc.

The cells may also be transformed or neoplastic cells of different types, e.g. carcinomas of different cell origins, lymphomas of different cell types, etc. The American Type Culture Collection (Manassas, VA) has collected and makes available over 4,000 cell lines from over 150 different species, over 950 cancer cell lines including 700 human cancer cell lines. The National Cancer Institute has compiled clinical, biochemical and molecular data from a large panel of human tumor cell lines, these are available from ATCC or the NCI (Phelps et al. (1996) Journal of Cellular Biochemistry Supplement 24:32-91). Included are different cell lines derived spontaneously, or selected for desired growth or response characteristics from an individual cell line; and may include multiple cell lines derived from a similar tumor type but from distinct patients or sites.

Cells may be non-adherent, e.g. blood cells including monocytes, T cells, B-cells; tumor cells, etc., or adherent cells, e.g. epithelial cells, endothelial cells, neural cells, etc. In order to profile adherent cells, they may be dissociated from the substrate that they are adhered to, and from other cells, in a manner that maintains their ability to recognize and bind to probe molecules.

Such cells can be acquired from an individual using, e.g., a draw, a lavage, a wash, surgical dissection etc., from a variety of tissues, e.g., blood, marrow, a solid tissue (e.g., a solid tumor), ascites, by a variety of techniques that are known in the art. Cells may be obtained from fixed or unfixed, fresh or frozen, whole or disaggregated samples. Disaggregation of tissue may occur either mechanically or enzymatically using known techniques.

### Imaging

The methods disclosed include imaging the one or more hydrogel-embedded amplicons using any of a number of different types of microscopy, e.g., confocal microscopy, two-photon microscopy, light-field microscopy, intact tissue expansion microscopy, and/or CLARITY™-optimized light sheet microscopy (COLM).

Bright field microscopy is the simplest of all the optical microscopy techniques. Sample illumination is via transmitted white light, i.e. illuminated from below and observed from above. Limitations include low contrast of most biological samples and low apparent resolution due to the blur of out of focus material. The simplicity of the technique and the minimal sample preparation required are significant advantages.

In oblique illumination microscopy, the specimen is illuminated from the side. This gives the image a 3-dimensional appearance and can highlight otherwise invisible features. A more recent technique based on this method is Hoffmann's modulation contrast, a system found on inverted microscopes for use in cell culture. Though oblique illumination suffers from the same limitations as bright field microscopy (low contrast of many biological samples; low apparent resolution due to out of focus objects), it may highlight otherwise invisible structures.

Dark field microscopy is a technique for improving the contrast of unstained, transparent specimens. Dark field illumination uses a carefully aligned light source to minimize the quantity of directly-transmitted (unscattered) light entering the image plane, collecting only the light scattered by the sample. Dark field can dramatically improve image contrast (especially of transparent objects) while requiring little equipment setup or sample preparation. However, the technique suffers from low light intensity in final image of many biological samples, and continues to be affected by low apparent resolution.

Phase contrast is an optical microscopy illumination technique that converts phase shifts in light passing through a transparent specimen to brightness changes in the image. In other words, phase contrast shows differences in refractive index as difference in contrast. The phase shifts themselves are invisible to the human eye, but become visible when they are shown as brightness changes.

In differential interference contrast (DIC) microscopy, differences in optical density will show up as differences in relief. The system consists of a special prism (Nomarski prism, Wollaston prism) in the condenser that splits light in an ordinary and an extraordinary beam. The spatial difference between the two beams is minimal (less than the maximum resolution of the objective). After passage through the specimen, the beams are reunited by a similar prism in the objective. In a homogeneous specimen, there is no difference between the two beams, and no contrast is being generated. However, near a refractive boundary (e.g. a nucleus within the cytoplasm), the difference between the ordinary and the extraordinary beam will generate a relief in the image. Differential interference contrast requires a polarized light source to function; two polarizing filters have to be fitted in the light path, one below the condenser (the polarizer), and the other above the objective (the analyzer).

Another microscopic technique using interference is interference reflection microscopy (also known as reflected interference contrast, or RIC). It is used to examine the adhesion of cells to a glass surface, using polarized light of a narrow range of wavelengths to be reflected whenever there is an interface between two substances with different refractive indices. Whenever a cell is attached to the glass surface, reflected light from the glass and that from the attached cell will interfere. If there is no cell attached to the glass, there will be no interference.

A fluorescence microscope is an optical microscope that uses fluorescence and phosphorescence instead of, or in addition to, reflection and absorption to study properties of organic or inorganic substances. In fluorescence microscopy, a sample is illuminated with light of a wavelength which excites fluorescence in the sample. The fluoresced light, which is usually at a longer wavelength than the illumination, is then imaged through a microscope objective. Two filters may be used in this technique; an illumination (or excitation) filter which ensures the illumination is near monochromatic and at the correct wavelength, and a second emission (or barrier) filter which ensures none of the excitation light source reaches the detector. Alternatively, these functions may both be accomplished by a single dichroic filter. The “fluorescence microscope” refers to any microscope that uses fluorescence to generate an image, whether it is a more simple set up like an epifluorescence microscope, or a more complicated design such as a confocal microscope, which uses optical sectioning to get better resolution of the fluorescent image.

Confocal microscopy uses point illumination and a pinhole in an optically conjugate plane in front of the detector to eliminate out-of-focus signal. As only light produced by fluorescence very close to the focal plane can be detected, the image's optical resolution, particularly in the sample depth direction, is much better than that of wide-field microscopes. However, as much of the light from sample fluorescence is blocked at the pinhole, this increased resolution is at the cost of decreased signal intensity—so long exposures are often required. As only one point in the sample is illuminated at a time, 2D or 3D imaging requires scanning over a regular raster (i.e., a rectangular pattern of parallel scanning lines) in the specimen. The achievable thickness of the focal plane is defined mostly by the wavelength of the used light divided by the numerical aperture of the objective lens, but also by the optical properties of the specimen. The thin optical sectioning possible makes these types of microscopes particularly good at 3D imaging and surface profiling of samples. COLM provides an alternative microscopy for fast 3D imaging of large clarified samples. COLM interrogates large immunostained tissues, permits increased speed of acquisition and results in a higher quality of generated data.

In single plane illumination microscopy (SPIM), also known as light sheet microscopy, only the fluorophores in the focal plane of the detection objective lens are illuminated. The light sheet is a beam that is collimated in one and focused in the other direction. Since no fluorophores are excited outside the detectors' focal plane, the method also provides intrinsic optical sectioning. Moreover, when compared to conventional microscopy, light sheet methods exhibit reduced photobleaching and lower phototoxicity, and often enable far more scans per specimen. By rotating the specimen, the technique can image virtually any plane with multiple views obtained from different angles. For every angle, however, only a relatively shallow section of the specimen is imaged with high resolution, whereas deeper regions appear increasingly blurred.

Super-resolution microscopy is a form of light microscopy. Due to the diffraction of light, the resolution of conventional light microscopy is limited as stated by Ernst Abbe in 1873. A good approximation of the resolution attainable is the FWHM (full width at half-maximum) of the point spread function, and a precise widefield microscope with high numerical aperture and visible light usually reaches a resolution of ˜250 nm. Super-resolution techniques allow the capture of images with a higher resolution than the diffraction limit. They fall into two broad categories, “true” super-resolution techniques, which capture information contained in evanescent waves, and “functional” super-resolution techniques, which use experimental techniques and known limitations on the matter being imaged to reconstruct a super-resolution image.

Laser microscopy uses laser illumination sources in various forms of microscopy. For instance, laser microscopy focused on biological applications uses ultrashort pulse lasers, or femtosecond lasers, in a number of techniques including nonlinear microscopy, saturation microscopy, and multiphoton fluorescence microscopy such as two-photon excitation microscopy (a fluorescence imaging technique that allows imaging of living tissue up to a very high depth, e.g. one millimeter)

In electron microscopy (EM), a beam of electrons is used to illuminate a specimen and produce a magnified image. An electron microscope has greater resolving power than a light-powered optical microscope because electrons have wavelengths about 100,000 times shorter than visible light (photons). They can achieve better than 50 pm resolution and magnifications of up to about 10,000,000× whereas ordinary, non-confocal light microscopes are limited by diffraction to about 200 nm resolution and useful magnifications below 2000×. The electron microscope uses electrostatic and electromagnetic “lenses” to control the electron beam and focus it to form an image. These lenses are analogous to but different from the glass lenses of an optical microscope that form a magnified image by focusing light on or through the specimen. Electron microscopes are used to observe a wide range of biological and inorganic specimens including microorganisms, cells, large molecules, biopsy samples, metals, and crystals. Industrially, the electron microscope is often used for quality control and failure analysis. Examples of electron microscopy include Transmission electron microscopy (TEM), Scanning electron microscopy (SEM), reflection electron microscopy (REM), Scanning transmission electron microscopy (STEM) and low-voltage electron microscopy (LVEM).

Scanning probe microscopy (SPM) is a branch of microscopy that forms images of surfaces using a physical probe that scans the specimen. An image of the surface is obtained by mechanically moving the probe in a raster scan of the specimen, line by line, and recording the probe-surface interaction as a function of position. Examples of SPM include atomic force microscopy (ATM), ballistic electron emission microscopy (BEEM), chemical force microscopy (CFM), conductive atomic force microscopy (C-AFM), electrochemical scanning tunneling microscope (ECS™), electrostatic force microscopy (EFM), fluidic force microscope (FluidFM), force modulation microscopy (FMM), feature-oriented scanning probe microscopy (FOSPM), kelvin probe force microscopy (KPFM), magnetic force microscopy (MFM), magnetic resonance force microscopy (MRFM), near-field scanning optical microscopy (NSOM) (or SNOM, scanning near-field optical microscopy, SNOM, Piezoresponse Force Microscopy (PFM), PSTM, photon scanning tunneling microscopy (PSTM), PTMS, photothermal microspectroscopy/microscopy (PTMS), SCM, scanning capacitance microscopy (SCM), SECM, scanning electrochemical microscopy (SECM), SGM, scanning gate microscopy (SGM), SHPM, scanning Hall probe microscopy (SHPM), SICM, scanning ion-conductance microscopy (SICM), SPSM spin polarized scanning tunneling microscopy (SPSM), SSRM, scanning spreading resistance microscopy (SSRM), SThM, scanning thermal microscopy (SThM), STM, scanning tunneling microscopy (STM), STP, scanning tunneling potentiometry (STP), SVM, scanning voltage microscopy (SVM), and synchrotron x-ray scanning tunneling microscopy (SXSTM).

Intact tissue expansion microscopy (exM) enables imaging of thick preserve specimens with roughly 70 nm lateral resolution. Using ExM the optical diffraction limit is circumvented by physically expanding a biological specimen before imaging, thus bringing sub-diffraction limited structures into the size range viewable by a conventional diffraction-limited microscope. ExM can image biological specimens at the voxel rates of a diffraction limited microscope, but with the voxel sizes of a super-resolution microscope. Expanded samples are transparent, and index-matched to water, as the expanded material is >99% water. Techniques of expansion microscopy are known in the art, e.g., as disclosed in Gao et al., Q&A: Expansion Microscopy, BMC Biol. 2017; 15:50.

### Screening Methods

The methods disclosed herein also provide for a method of screening a candidate agent to determine whether the candidate agent modulates gene expression of a nucleic acid in a cell in an intact tissue. The method comprises performing the steps of STARmap2 disclosed herein to determine the gene sequence of a target nucleic acid in the cell in an intact tissue, and detecting the level of gene expression of the target nucleic acid, wherein an alteration in the level of expression of the target nucleic acid in the presence of the candidate agent relative to the level of expression of the target nucleic acid in the absence of the candidate agent indicates that the candidate agent modulates gene expression of the nucleic acid in the cell in the intact tissue.

In some aspects, the detecting includes performing flow cytometry; sequencing; probe binding and electrochemical detection; pH alteration; catalysis induced by enzymes bound to DNA tags; quantum entanglement; Raman spectroscopy; terahertz wave technology; and/or scanning electron microscopy. In certain aspects, the flow cytometry is mass cytometry or fluorescence-activated flow cytometry. In some other aspects, the detecting includes performing microscopy, scanning mass spectrometry or other imaging techniques described herein. In such aspects, the detecting includes determining a signal, e.g., a fluorescent signal.

By “test agent,” “candidate agent,” and grammatical equivalents herein, which terms are used interchangeably herein, is meant any molecule (e.g. proteins (which herein includes proteins, polypeptides, and peptides), small (i.e., 5-1000 Da, 100-750 Da, 200-500 Da, or less than 500 Da in size), or organic or inorganic molecules, polysaccharides, polynucleotides, etc.) which are to be tested for activity in a subject assay.

A variety of different candidate agents may be screened by the above methods. Candidate agents encompass numerous chemical classes, e.g., small organic compounds having a molecular weight of more than 50 daltons (e.g., at least about 50 Da, at least about 100 Da, at least about 150 Da, at least about 200 Da, at least about 250 Da, or at least about 500 Da) and less than about 20,000 daltons, less than about 10,000 daltons, less than about 5,000 daltons, or less than about 2,500 daltons. For example, in some embodiments, a suitable candidate agent is an organic compound having a molecular weight in a range of from about 500 Da to about 20,000 Da, e.g., from about 500 Da to about 1000 Da, from about 1000 Da to about 2000 Da, from about 2000 Da to about 2500 Da, from about 2500 Da to about 5000 Da, from about 5000 Da to about 10,000 Da, or from about 10,000 Da to about 20,000 Da.

Candidate agents can include functional groups necessary for structural interaction with proteins, e.g., hydrogen bonding, and can include at least an amine, carbonyl, hydroxyl or carboxyl group, or at least two of the functional chemical groups. The candidate agents can include cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof.

Candidate agents are obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs. Moreover, screening may be directed to known pharmacologically active compounds and chemical analogs thereof, or to new agents with unknown properties such as those created through rational drug design.

In one embodiment, candidate modulators are synthetic compounds. Any number of techniques is available for the random and directed synthesis of a wide variety of organic compounds and biomolecules, including expression of randomized oligonucleotides. See for example WO 94/24314, hereby expressly incorporated by reference, which discusses methods for generating new compounds, including random chemistry methods as well as enzymatic methods.

In another embodiment, the candidate agents are provided as libraries of natural compounds in the form of bacterial, fungal, plant and animal extracts that are available or readily produced. Additionally, natural or synthetically produced libraries and compounds are readily modified through conventional chemical, physical and biochemical means. Known pharmacological agents may be subjected to directed or random chemical modifications, including enzymatic modifications, to produce structural analogs.

In one embodiment, candidate agents include proteins (including antibodies, antibody fragments (i.e., a fragment containing an antigen-binding region, single chain antibodies, and the like), nucleic acids, and chemical moieties. In one embodiment, the candidate agents are naturally occurring proteins or fragments of naturally occurring proteins. Thus, for example, cellular extracts containing proteins, or random or directed digests of proteinaceous cellular extracts, may be tested. In this way libraries of prokaryotic and eukaryotic proteins may be made for screening. Other embodiments include libraries of bacterial, fungal, viral, and mammalian proteins (e.g., human proteins).

In one embodiment, the candidate agents are organic moieties. In this embodiment, as is generally described in WO 94/243 14, candidate agents are synthesized from a series of substrates that can be chemically modified. “Chemically modified” herein includes traditional chemical reactions as well as enzymatic reactions. These substrates generally include, but are not limited to, alkyl groups (including alkanes, alkenes, alkynes and heteroalkyl), aryl groups (including arenes and heteroaryl), alcohols, ethers, amines, aldehydes, ketones, acids, esters, amides, cyclic compounds, heterocyclic compounds (including purines, pyrimidines, benzodiazepins, beta-lactams, tetracylines, cephalosporins, and carbohydrates), steroids (including estrogens, androgens, cortisone, ecodysone, etc.), alkaloids (including ergots, vinca, curare, pyrollizdine, and mitomycines), organometallic compounds, hetero-atom bearing compounds, amino acids, and nucleosides. Chemical (including enzymatic) reactions may be done on the moieties to form new substrates or candidate agents which can then be tested using the present invention.

### Devices and Systems

Also included are devices for performing aspects of the subject methods. The subject devices may include, for example, imaging chambers, electrophoresis apparatus, flow chambers, microscopes, needles, tubing, pumps.

The present disclosure also provides systems for performing the subject methods. Systems may include, e.g. a power supply, a refrigeration unit, waste, a heating unit, a pump, etc. Systems may also include any of the reagents described herein, e.g. imaging buffer, wash buffer, strip buffer, Nissl and DAPI solutions. Systems in accordance with certain embodiments may also include a microscope and/or related imaging equipment, e.g., camera components, digital imaging components and/or image capturing equipment, computer processors configured to collect images according to one or more user inputs, and the like.

As discussed above, the systems described herein include a fluidics device having an imaging chamber and a pump; and a processor unit configured to perform the methods for in situ gene sequencing of a target nucleic acid in a cell in an intact tissue described herein. In some embodiments, the system enables the automation of STARmap2, a process described herein as including, but not limited to, repeated rounds of hybridization of probes with DNA embedded in a gel, ligation of fluorescently labeled oligonucleotides onto these probes, washing off the excess probes, imaging, and stripping off the probes for the next round of sequencing. In some embodiments, the system may allow for continual operation. In some embodiments, the system includes an imaging chamber for flowing sequencing chemicals involved in in situ DNA sequencing over a sample. In some embodiments, the system of fluidics and pumps control sequencing chemical delivery to the sample.

Buffers may be added/removed/recirculated/replaced by the use of the one or more ports and optionally, tubing, pumps, valves, or any other suitable fluid handling and/or fluid manipulation equipment, for example, tubing that is removably attached or permanently attached to one or more components of a device. For example, a first tube having a first and second end may be attached to a first port and a second tube having a first and second end may be attached to a second port, where the first end of the first tube is attached to the first port and the second end of the first tube is operably linked to a receptacle, e.g. a cooling unit, heating unit, filtration unit, waste receptacle, etc.; and the first end of the second tube is attached to the second port and the second end of the second tube is operably linked to a receptacle, e.g. a cooling unit, beaker on ice, filtration unit, waste receptacle, etc.

In some embodiments, the system includes a non-transitory computer-readable storage medium that has instructions, which when executed by the processor unit, cause the processor unit to control the delivery of chemicals and synchronize this process with a microscope. In some embodiments, the non-transitory computer-readable storage medium includes instructions, which when executed by the processor unit, cause the processor unit to measure an optical signal.

### Utility

The devices, methods, and systems herein find a number of uses in the art such as in biomedical research and/or clinical diagnostics. For example, in biomedical research, applications include, but are not limited to, spatially resolved gene expression analysis for fundamental biology or drug screening. In clinical diagnostics, applications include, but are not limited to, detecting gene markers such as disease, immune responses, bacterial or viral DNA/RNA for patient samples. Examples of advantages of the methods described herein include efficiency, where it takes merely 3 or 4 days to obtain final data from a raw sample, providing speeds much faster than existing microarray or sequencing technology; highly multiplexed (up to 1000 genes); single-cell and single-molecule sensitivity; preserved tissue morphology; and/or high signal-to-noise ratio with low error rates.

In certain aspects, STARmap2 may be applied to the study of molecular-defined cell types and activity-regulated gene expression in mouse visual cortex, and to be scalable to larger 3D tissue blocks to visualize short- and long-range spatial organization of cortical neurons on a volumetric scale not previously accessible. In some embodiments, the methods disclosed herein may be adapted to image DNA-conjugated antibodies for highly multiplexed protein detection.

The devices, methods, and systems of the invention can also be generalized to study a number of heterogeneous cell populations in diverse tissues. Without being bound by any scientific theory, the brain poses special challenges well suited to STARmap2 analysis. For example, the polymorphic activity-regulated gene (ARG) expression observed across different cell types is likely to depend on both intrinsic cell-biological properties (such as signal transduction pathway-component expression), and on extrinsic properties such as neural circuit anatomy that routes external sensory information to different cells (here in visual cortex). In such cases, in situ transcriptomics exemplified by STARmap2 can effectively link imaging-based molecular information with anatomical and activity information, thus elucidating brain function and dysfunction.

The devices, methods, and systems disclosed herein enable cellular components, e.g. lipids that normally provide structural support but that hinder visualization of subcellular proteins and molecules to be removed while preserving the 3-dimensional architecture of the cells and tissue because the sample is crosslinked to a hydrogel that physically supports the ultrastructure of the tissue. This removal renders the interior of biological specimen substantially permeable to light and/or macromolecules, allowing the interior of the specimen, e.g. cells and subcellular structures, to be microscopically visualized without time-consuming and disruptive sectioning of the tissue. The procedure is also more rapid than procedures commonly used in the art, as clearance and permeabilization, typically performed in separate steps, may be combined in a single step of removing cellular components. Additionally, the specimen can be iteratively stained, unstained, and re-stained with other reagents for comprehensive analysis. Further functionalization with the polymerizable acrylamide moiety enables amplicons to be covalently anchored within the polyacrylamide network at multiple sites.

In one example, the subject devices, methods, and systems may be employed to evaluate, diagnose or monitor a disease. “Diagnosis” as used herein generally includes a prediction of a subject's susceptibility to a disease or disorder, determination as to whether a subject is presently affected by a disease or disorder, prognosis of a subject affected by a disease or disorder (e.g., identification of cancerous states, stages of cancer, likelihood that a patient will die from the cancer), prediction of a subject's responsiveness to treatment for a disease or disorder (e.g., a positive response, a negative response, no response at all to, e.g., allogeneic hematopoietic stem cell transplantation, chemotherapy, radiation therapy, antibody therapy, small molecule compound therapy) and use of therametrics (e.g., monitoring a subject's condition to provide information as to the effect or efficacy of therapy). For example, a biopsy may be prepared from a cancerous tissue and microscopically analyzed to determine the type of cancer, the extent to which the cancer has developed, whether the cancer will be responsive to therapeutic intervention, etc.

The subject devices, methods, and systems also provide a useful technique for screening candidate therapeutic agents for their effect on a tissue or a disease. For example, a subject, e.g. a mouse, rat, dog, primate, human, etc. may be contacted with a candidate agent, an organ or a biopsy thereof may be prepared by the subject methods, and the prepared specimen microscopically analyzed for one or more cellular or tissue parameters. Parameters are quantifiable components of cells or tissues, particularly components that can be accurately measured, desirably in a high throughput system. A parameter can be any cell component or cell product including cell surface determinant, receptor, protein or conformational or posttranslational modification thereof, lipid, carbohydrate, organic or inorganic molecule, nucleic acid, e.g. mRNA, DNA, etc. or a portion derived from such a cell component or combinations thereof. While most parameters will provide a quantitative readout, in some instances a semi-quantitative or qualitative result will be acceptable. Readouts may include a single determined value, or may include mean, median value or the variance, etc. Characteristically a range of parameter readout values will be obtained for each parameter from a multiplicity of the same assays. Variability is expected and a range of values for each of the set of test parameters will be obtained using standard statistical methods with a common statistical method used to provide single values. Thus, for example, one such method may include detecting cellular viability, tissue vascularization, the presence of immune cell infiltrates, efficacy in altering the progression of the disease, etc. In some embodiments, the screen includes comparing the analyzed parameter(s) to those from a control, or reference, sample, e.g., a specimen similarly prepared from a subject not contacted with the candidate agent. Candidate agents of interest for screening include known and unknown compounds that encompass numerous chemical classes, primarily organic molecules, which may include organometallic molecules, inorganic molecules, genetic sequences, etc. Candidate agents of interest for screening also include nucleic acids, for example, nucleic acids that encode siRNA, shRNA, antisense molecules, or miRNA, or nucleic acids that encode polypeptides. An important aspect of the invention is to evaluate candidate drugs, including toxicity testing; and the like. Evaluations of tissue samples using the subject methods may include, e.g., genetic, transcriptomic, genomic, proteomic, and/or metabolomics analyses.

The subject devices, methods, and systems may also be used to visualize the distribution of genetically encoded markers in whole tissue at subcellular resolution, for example, chromosomal abnormalities (inversions, duplications, translocations, etc.), loss of genetic heterozygosity, the presence of gene alleles indicative of a predisposition towards disease or good health, likelihood of responsiveness to therapy, ancestry, and the like. Such detection may be used in, for example, diagnosing and monitoring disease as, e.g., described above, in personalized medicine, and in studying paternity.

A database of analytic information can be compiled. These databases may include results from known cell types, references from the analysis of cells treated under particular conditions, and the like. A data matrix may be generated, where each point of the data matrix corresponds to a readout from a cell, where data for each cell may include readouts from multiple labels. The readout may be a mean, median or the variance or other statistically or mathematically derived value associated with the measurement. The output readout information may be further refined by direct comparison with the corresponding reference readout. The absolute values obtained for each output under identical conditions will display a variability that is inherent in live biological systems and also reflects individual cellular variability as well as the variability inherent between individuals. Examples of Non-Limiting Aspects of the Disclosure

Aspects, including embodiments, of the present subject matter described above may be beneficial alone or in combination, with one or more other aspects or embodiments. Without limiting the foregoing description, certain non-limiting aspects of the disclosure numbered 1-71 are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:

1. A method for in situ gene sequencing of a target nucleic acid in a cell in an intact tissue, the method comprising:
(a) contacting a fixed and permeabilized intact tissue with at least a pair of oligonucleotide primers under conditions to allow for specific hybridization,
wherein the pair of primers comprise a first oligonucleotide and a second oligonucleotide;
wherein each of the first oligonucleotide and the second oligonucleotide comprises a first complementarity region, a second complementarity region sequence, and a third complementarity region; wherein the second oligonucleotide further comprises a barcode sequence;
wherein the first complementarity region of the first oligonucleotide is complementary to a first portion of the target nucleic acid, wherein the second complementarity region of the first oligonucleotide is complementary to the first complementarity region of the second oligonucleotide,
wherein the third complementarity region of the first oligonucleotide is complementary to the third complementarity region of the second oligonucleotide, wherein the second complementary region of the second oligonucleotide is complementary to a second portion of the target nucleic acid,
wherein the first portion of the target nucleic is adjacent to the second portion of the target nucleic acid, wherein the second complementarity region of the first oligonucleotide comprises a first portion of a unique matching sequence and the third complementarity region of the first oligonucleotide comprises a second portion of the unique matching sequence, and the first complementarity region of the second oligonucleotide comprises a sequence that is complementary to the first portion of the unique matching sequence, and the third complementarity region of the second oligonucleotide comprises a sequence that is complementary to the second portion of the unique matching sequence;
(b) adding ligase to ligate the second oligonucleotide and generate a closed nucleic acid circle;
(c) performing rolling circle amplification in the presence of a nucleic acid molecule, wherein the performing comprises using the second oligonucleotide as a template and the first oligonucleotide as a primer for a polymerase to form one or more amplicons;
(d) embedding the one or more amplicons in the presence of hydrogel subunits to form one or more hydrogel-embedded amplicons;
(e) contacting the one or more hydrogel-embedded amplicons having the barcode sequence with a set of sequencing primers under conditions to allow for ligation, wherein the set of sequencing primers comprises a third oligonucleotide configured to decode bases and a fourth oligonucleotide configured to convert decoded bases into a signal, wherein the ligation only occurs when both the third oligonucleotide and the fourth oligonucleotide are complementary to adjacent sequences of the same amplicon;
(f) reiterating step (e); and
(g) imaging the one or more hydrogel-embedded amplicons to determine in situ a gene sequence of the target nucleic acid in the cell in the intact tissue.

2. The method of aspect 1 or 2, further comprising contacting the fixed and permeabilized intact tissue with a gel adaptor oligonucleotide that binds to the first oligonucleotide, wherein the gel adaptor oligonucleotide comprises a nucleotide modification at the 5′ end or the 3′ end that links the gel adapter to the hydrogel during gelation.

3. The method of aspect 2, wherein the modification comprises an acrydite group.

4. The method of aspect 2 or 3, wherein the first oligonucleotide further comprises a common binding site for the gel adaptor oligonucleotide.

5. The method of aspect 4, wherein the common binding site for the gel adaptor oligonucleotide is adjacent to the first complementarity region of the first oligonucleotide.

6. The method of any one of aspects 1-5, further comprising contacting the fixed and permeabilized intact tissue with an oligonucleotide probe for detection and condensing of the amplicons, wherein the oligonucleotide probe binds to the second oligonucleotide.

7. The method of aspect 6, wherein the second oligonucleotide further comprises a common binding site for the oligonucleotide probe for detection and condensing of the amplicons.

8. The method of aspect 7, wherein the common binding site for the oligonucleotide probe for detection and condensing of the amplicons is adjacent to the second complementarity region or adjacent to the sequence that is complementary to the second half of the unique matching sequence of the second oligonucleotide.

9. The method of any one of aspects 6-8, wherein the oligonucleotide probe comprises a unique sequence for detecting an amplicon of a probe target of interest and two or more copies of a sequence complementary to a common sequence on the amplicons.

10. The method of any one of aspects 6-9, wherein the oligonucleotide probe further comprises a nucleotide modification at the 5′ end or 3′ end such that the first oligonucleotide probe is linked to the hydrogel during gelation.

11. The method of aspect 10, wherein the modification comprises an acrydite group.

12. The method of any one of aspects 1-11, further comprising barcoding a cell by contacting the cell with:
a first probe comprising a 5′-amine modification or a 5′-biotin modification, a common gel adaptor complementary sequence that hybridizes with the gel adaptor oligonucleotide, and a unique barcode sequence; and
a second probe comprising a first sequence that is complementary to a first portion of the unique barcode sequence and a second sequence that is complementary to a second portion of the unique barcode sequence, wherein the first sequence and the second sequence flank a sequencing encoding sequence, wherein hybridization of the first probe and the second probe results in formation of a barcoding complex comprising the first probe and the second probe.

13. The method of aspect 12, wherein the second probe is a padlock probe.

14. The method of any one of aspects 1-13, wherein the first portion of the target nucleic acid and the second portion of the target nucleic acid have about the same melting temperature.

15. The method of any one of aspects 1-14, further comprising contacting the fixed and permeabilized intact tissue with an mRNA retention oligonucleotide, wherein the mRNA retention oligonucleotide comprises:
a nucleotide modification at the 5′ end or 3′ end, wherein the mRNA retention oligonucleotide is linked to the hydrogel during gelation;
a poly-T tail that hybridizes to a poly-A tail of an mRNA, wherein hybridization of the poly-T tail of the mRNA retention oligonucleotide to the poly-A tail of the mRNA retains the mRNA in the hydrogel; and
a unique hybridization sequence.

16. The method of aspect 15, wherein the poly-T tail comprises interleaved locked nucleic acid (LNA) thymine (T) bases.

17. The method of aspect 15 or 16, further comprising contacting the fixed and permeabilized intact tissue with a fluorescently labeled probe oligonucleotide that selectively binds to the unique hybridization sequence of the mRNA retention oligonucleotide.

18. The method of any one of aspects 1-17, wherein sequencing is performed with sequential or combinatorial encoding.

19. The method of any one of aspects 1-18, further comprising preincubating the tissue sample with the polymerase for a sufficient time to allow uniform diffusion of the polymerase throughout the tissue before performing the rolling circle amplification.

20. The method of any one of aspects 1-19, wherein said imaging is performed in presence of an anti-fade buffer comprising an antioxidant.

21. The method of aspect 20, wherein the anti-fade buffer comprises trolox (6-hydroxy-2,5,7,8-tetramethylchroman-2-carboxylic acid) and trolox-quinone.

22. The method of any one of aspects 1-21, wherein the signal is a fluorescent signal.

23. The method of aspect 22, further comprising removing the signal after imaging by contacting the hydrogel with formamide.

24. The method of aspect 22, wherein the fourth oligonucleotide is covalently linked to a fluorophore by a disulfide bond.

25. The method of aspect 24, further comprising contacting the hydrogel with a reducing agent after said imaging, wherein reduction of the disulfide bond results in cleavage of the fluorophore from the fourth oligonucleotide.

26. The method of any one of aspects 1-25, wherein the set of primers are denatured by heating before contacting the sample.

27. The method of any one of aspects 1-26, wherein the cell is present in a population of cells.

28. The method of aspect 27, wherein the population of cells comprises a plurality of cell types.

29. The method of any one of aspects 1-28, wherein the contacting the fixed and permeabilized intact tissue comprises hybridizing the primers to the same target nucleic acid.

30. The method of any one of aspects 1-29, wherein the target nucleic acid is RNA or DNA.

31. The method of aspect 30, wherein the RNA is mRNA.

32. The method of any one of aspects 1-31, wherein the second oligonucleotide comprises a padlock probe.

33. The method of any one of aspects 1-32, wherein the first complementarity region of the first oligonucleotide has a length of 19-25 nucleotides.

34. The method of any one of aspects 1-33, wherein the second complementarity region of the first oligonucleotide has a length of 6 nucleotides.

35. The method of any one of aspects 1-34, wherein the third complementarity region of the first oligonucleotide has a length of 6 nucleotides.

36. The method of any one of aspects 1-35, wherein the first complementarity region of the second oligonucleotide has a length of 6 nucleotides.

37. The method of any one of aspects 1-36, wherein the second complementarity region of the second oligonucleotide has a length of 19-25 nucleotides.

38. The method of any one of aspects 1-37, wherein the third complementarity region of the second oligonucleotide has a length of 6 nucleotides.

39. The method of any one of aspects 1-38, wherein the first complementarity region of the second oligonucleotide comprises the 5′ end of the second oligonucleotide.

40. The method of any one of aspects 1-39, wherein the third complementarity region of the second oligonucleotide comprises the 3′ end of the second oligonucleotide.

41. The method of any one of aspects 1-40, wherein the first complementarity region of the second oligonucleotide is adjacent to the third complementarity region of the second oligonucleotide.

42. The method of any one of aspects 1-41, wherein the barcode sequence of the second oligonucleotide provides barcoding information for identification of the target nucleic acid.

43. The method of any one of aspects 1-42, wherein the contacting the fixed and permeabilized intact tissue comprises hybridizing a plurality of oligonucleotide primers having specificity for different target nucleic acids.

44. The method of any one of aspects 1-43, wherein the second oligonucleotide is provided as a closed nucleic acid circle, and the step of adding ligase is omitted.

45. The method of any of aspects 1-44, wherein the melting temperature (Tₘ) of oligonucleotides is selected to minimize ligation in solution.

46. The method of any one of aspects 1-45, wherein the adding ligase comprises adding a DNA ligase.

47. The method of any one of aspects 1-46, wherein the nucleic acid molecule comprises an amine-modified nucleotide.

48. The method of aspect 47, wherein the amine-modified nucleotide comprises an acrylic acid N-hydroxysuccinimide moiety modification.

49. The method of any one of aspects 1-48, wherein the embedding comprises copolymerizing the one or more amplicons with acrylamide.

50. The method of any one of aspects 1-49, wherein the embedding comprises clearing the one or more hydrogel-embedded amplicons wherein the target nucleic acid is substantially retained in the one or more hydrogel-embedded amplicons.

51. The method of aspect 50, wherein the clearing comprises substantially removing a plurality of cellular components from the one or more hydrogel-embedded amplicons.

52. The method of aspect 50 or 51, wherein the clearing comprises substantially removing lipids or proteins, or a combination thereof from the one or more hydrogel-embedded amplicons.

53. The method of any one of aspects 1-52, wherein the contacting the one or more hydrogel-embedded amplicons comprises eliminating error accumulation as sequencing proceeds.

54. The method of any one of aspects 1-53, wherein the imaging comprises imaging the one or more hydrogel-embedded amplicons using confocal microscopy, two-photon microscopy, light-field microscopy, intact tissue expansion microscopy, and/or CLARITY™-optimized light sheet microscopy (COLM).

55. The method of any one of aspects 1-54, wherein the intact tissue is a thin slice.

56. The method of aspect 55, wherein the intact tissue has a thickness of 5-20 μm.

57. The method of aspect 55 or 56, wherein the contacting the one or more hydrogel-embedded amplicons occurs four times or more.

58. The method of any one of aspects 1-54, wherein the intact tissue is a thick slice.

59. The method of aspect 58, wherein the intact tissue has a thickness of 50-200 μm.

60. The method of aspect 58 or 59, wherein the contacting the one or more hydrogel-embedded amplicons occurs six times or more.

61. The method of any one of aspects 1-60, wherein the ligation of the third oligonucleotide and the fourth oligonucleotide is performed in presence of a polyethylene glycol polymer.

62. The method of aspect 61, wherein the PEG polymer is PEG 6000.

63. A method of screening a candidate agent to determine whether the candidate agent modulates gene expression of a nucleic acid in a cell in an intact tissue, the method comprising performing the method of any one of aspects 1-62 to determine the gene sequence of the target nucleic acid in the cell in the intact tissue, and detecting the level of gene expression of the target nucleic acid, wherein an alteration in the level of expression of the target nucleic acid in the presence of the candidate agent relative to the level of expression of the target nucleic acid in the absence of the candidate agent indicates that the candidate agent modulates gene expression of the nucleic acid in the cell in the intact tissue.

64. The method of aspect 63, wherein the detecting comprises performing flow cytometry; sequencing; probe binding and electrochemical detection; pH alteration; catalysis induced by enzymes bound to DNA tags; quantum entanglement; Raman spectroscopy; terahertz wave technology; and/or scanning electron microscopy.

65. The method of aspect 64, wherein the flow cytometry is mass cytometry or fluorescence-activated flow cytometry.

66. The method of any one of aspects 63-65, wherein the detecting comprises performing microscopy, scanning mass spectrometry, or other imaging techniques

67. The method of any one of aspects 63-66, wherein the detecting comprises detecting a signal.

68. The method of aspect 67, wherein the signal is a fluorescent signal.

69. A system, comprising:
a fluidics device, and a processor unit configured to perform the method of any one of aspects 1-68.

70. The system of aspect 69, further comprising an imaging chamber.

71. The system of aspect 69 or 70, further comprising a pump.

### Experimental

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

All publications and patent applications cited in this specification are herein incorporated by reference as if each individual publication or patent application were specifically and individually indicated to be incorporated by reference.

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. For example, due to codon redundancy, changes can be made in the underlying DNA sequence without affecting the protein sequence. Moreover, due to biological functional equivalency considerations, changes can be made in protein structure without affecting the biological action in kind or amount. All such modifications are intended to be included within the scope of the appended claims.

### Example 1

### STARmap2 Next-Generation Volumetric In Situ Sequencing

### Introduction

STARmap is a sequencing process in which target nucleic acids are selectively labeled with barcodes, which are amplified and affixed into a hydrogel before subsequent multi-round readout by ligation. The original publication of STARmap demonstrated the usage of combinatorial sequencing on sample sizes as large as 0.0034 cubic millimeters and the usage of sequential sequencing on sample sizes as large as 0.238 cubic millimeters. Collecting these data required considerable human effort; the read-out stage of sequencing alone required multiple continuous days of effort for a single sample. An entire adult mouse brain is roughly 400 cubic millimeters, or more than 110,000× the volume of the previously demonstrated combinatorial sequencing sample. As intact-tissue sequencing approaches scale to increasing volumes and throughputs, maximizing their robustness (to prevent the compounding accumulation of error) and enhancing their parallelism and throughput is of critical importance. This is especially the case as these techniques move out of the lab and into clinical and industrial workflows.

### STARmap2 Next-Gen Volumetric In Situ Sequencing

1. Probe design

Background labeling reduction by gene or probe-pair specific matching signal scaffolding the ligation of each DNA part of the SNAIL complex
2. Adaptor oligos
3. Retention and labeling oligos
4. Chemical retention of targets
   Fixation and permeabilization conditions
   Other chemical modifications of targets
   Optional target retention into gel prior to hybridization and subsequent steps
5. Floating gel
6. Accelerated ligation with PEG
7. RCA conditions
8. Amplicon structuring and self-organization with linker oligos
9. Floating gel reattachment
10. Amplicon attachment
11. SCAL and SEDAL2 sequencing for STARmap2
   Reduced cycling time and simplified reagents for SCAL in STARmap2
   Forward and backward sequencing for high efficiency combinatorial barcode reads in STARmap2

### Technical Description

### Probe Design

### Targeted Probes—Complementary Sequence Identification

For a given species, a library of expressed genes was obtained by taking the shortest isoforms for each gene from a library of all cDNA sequences for that species. Using Bowtie2, a bowtie index was constructed from the shortest isoforms. Candidate complementary sequences were extracted using the blockParse.py functionality of OligoMiner in overlap mode with no temperature constraints, an oligo length between 44 and 48 nucleotide (nt), and a minimum GC content of 30% and a maximum GC content of 70%. Candidate complementary sequences were then aligned to the bowtie index to check for uniqueness, and all non-unique sequences were removed from the set of candidate oligos. Finally, starting from the first position per gene, overlapping oligos were removed from the set of candidate genes.

### Targeted Probes—SNAIL Construction

For a given set of gene names, a library of SNAIL probes was designed, taking as input the target number of SNAIL pairs per gene (typically 4 or 16) and whether encoding for sequencing should be sequential or combinatorial. For combinatorial encoded probe libraries, barcode sequences following a hamming encoding scheme were precomputed for a variety of code lengths and minimum distances, and based on the number of genes, an appropriate encoding set was chosen (for example, for the 200 gene set, a 7-long code with minimum distance 3 was used). Barcode sequences from this codebook were additionally filtered such that, with two-base encoding, they did not produce only a single color across all rounds. These filtered barcodes were assigned uniquely per gene. For both combinatorial and sequential encoding, probe sequences were then designed for each of the two SNAIL probe sequence parts, the head and shell. Additionally, rather than using a common sequence across all probes in the probe set for the two adjacent complementary sites at the 3′ end of the SNAIL head, as was done previously, a unique matching sequence (SNAIL match sequence), subject to GC constraints, was generated either per gene or per probe pair to be used for complementing each end of the SNAIL shell (where the ends hybridize together and are subsequently ligated). These SNAIL match sequences have the effect of minimizing spurious probe-probe interactions (as the ligase rejects mismatched sequences) that could occur either due to off-target hybridization, protein binding, or other probe aggregation or persistent close interaction, and thus facilitate specificity when greater numbers of genes are targeted or higher probe concentrations are used. For a given probe pair, a unique sequence of the targeted gene was selected, reverse complemented, and split into two parts, such that each part had a melting temperature approximately equal to the other part. A common 20 nt hybridization sequence (see Gel adaptor probe below) was followed by the first part of the reverse-complemented target gene sequence, followed by the probe-pair-specific or gene-specific SNAIL match sequence to form the SNAIL head. The second part of the reverse complement of the target sequence was added after the reverse complement of the first half of the SNAIL match sequence, followed by sequencing bases, and then the reverse complement of the second half of the SNAIL match sequence. For probe sets in which an amplicon condensing and detection oligo was used (see Thick section, library generation), an additional sequence, common for all SNAIL shells, was added to the SNAIL shell sequence, either adjacent to the target complementary sequence, or adjacent to the unique SNAIL match sequence, such that randomized N bases in the amplicon condensing and detection oligo tend to differentiate between hybridization targets (see Thick section, library generation).

### Gel Adaptor Oligo

An oligo complementary to the 5′ hybridization sequence on the SNAIL head was designed with functional attachment modification at its 5′ end, such as Acrydite, such that the SNAIL head sequence would be covalently linked to the hydrogel during gelation (thus better retaining the resulting amplicon grown from the 3′ end of this sequence), without requiring synthesis of the SNAIL head oligo with a 5′ modification itself.

mRNA Retention and Label Oligo

An mRNA retention and labeling oligo was designed with a 5′-Acrydite modification for retention in the hydrogel and a 14-mer poly-dt sequence containing interleaved locked nucleic acid (LNA) T bases (for stable hybridization to mRNA poly-A tails), followed by a unique 18 nt hybridization sequence. This oligo can be used to retain mRNA in the hydrogel during or prior to hybridization, additionally enabling gelation to be performed before hybridization in some cases. Importantly, the unique hybridization sequence on the oligo allows for labeling of total mRNA signal, even after RNA has been degraded, via hybridization with a complementary fluorophore labeled oligo. This is important for cytosolic-labeling based segmentation in the data processing pipeline following sequencing.

### Cell Barcoding

Per-cell barcodes were designed in two components. First, a 5′ splint sequence, containing either a 5′ amine modification (for fixation or subsequent modification) or a 5′ biotin modification (for facilitated polar trafficking through cells), the common gel adaptor complementary sequence, and a unique 40 nt sequence. Second, a padlock probe containing 20 nt of sequence at each end complementary to each half of the unique 40 nt sequence of the first probe, flanking a sequencing encoding sequence (for example, the sequential encoding sequence for a particular round and base). The pair of components, pre-hybridized together, constituted a barcode that could follow through the STARmap2 procedures of fixation, hybridization (with the gel adaptor oligo), polymerization into the hydrogel, ligation, and amplification by RCA, enabling STARmap2 read out of the encoded sequence along with any endogenous signals being detected. See STARpatch below for methodological description of an example use of these cell barcodes for cell tagging and morphological reconstruction following patch clamp recording in an intact tissue volume.

### STARmap2—Thin Section, Combinatorial and Sequential

### Sample Preparation

Mice were deeply anesthetized using 5% isofluorane and decapitated. Brains were rapidly dissected from the skull, immersed in OCT in chucks, flash-frozen in a slurry of ethanol and dry ice, and stored at −80° C. until use (minimum 30 minutes before use). Before sectioning, brains were equilibrated to cutting temperatures in a cryostat for at least 15 minutes. Sections were cut to a thickness of 16 m or less and transferred to wells of a glass-bottom plate which had been pre-treated with Bind-Silane and poly-L-lysine according to manufacturers' instructions. Following collection of tissue, sections were fixed for 10 minutes to 1 hour at room temperature (or up to 24 hours at 4° C.) in 4% paraformaldehyde (PFA). Sections were subsequently rinsed 3× with ice-cold phosphate-buffered saline (PBS) and transferred to methanol which had been pre-chilled to −20° C. Samples were stored for at least 15 minutes at −80° C. and maximally for several months before use.

### Sample Preparation—Slide Attached

Tissue previously collected onto a slide, with or without fixation, was prepared subsequently as followed. If tissue is FFPE and sectioned onto a slide, standard paraffin removal with xylene, washes with ethanol, and sample rehydration are performed as typical for FFPE sample recovery. If sample is slide attached but not fixed, samples are briefly fixed with 4% PFA at room temperature before proceeding. A hydrophobic barrier pen is used to draw a well around each sample on the glass slide, and subsequent sample preparation steps are performed within this well, according to the library generation procedure as outlined below. In cases where the sample is over-fixed and no longer penetrable by enzyme, the rehydration and hybridization and wash steps below can be first performed, followed by hydrogel embedding and clearing, and subsequent separation of the hydrogel from the glass slide (as the proteinase has destroyed the attachment of the tissue to the glass) with a razor or other implement. The ligation, amplification, and re-embedding steps are subsequently performed in a manner similar to the thick section protocol below.

### Library Generation

Sections were equilibrated to room temperature and rehydrated in PBS containing 0.1% Tween-20 plus 0.1 U/μl Superase RNAse inhibitor (PBSTwR) for at least 20 minutes, followed by a second rinse with PBSTwR to remove any residual methanol. Samples were placed into hybridization buffer containing 2× saline-sodium citrate (SSC) buffer, 10% formamide, 0.1% Tween-20, 0.1 mg/mL sheared salmon sperm DNA, probe sets appropriate for combinatorial or sequential sequencing (see Targeted probes) at a final concentration of between 1 nM and 100 nM per probe pair (probe aliquot heated to 90° C. for 3 minutes and allowed to cool slowly to room temperature prior to addition to hybridization buffer), mRNA-label oligo at 3 uM, Superase RNAse inhibitor at a concentration of 0.2 U/μl, RNAse-free distilled and deionized water (H₂O), and sterile 10% Dextran-sulfate solution. In some cases the Dextran-sulfate solution was omitted. At least 50 μl of hybridization buffer was added to samples and typically between 125 μl and 250 μl. Samples were incubated, agitated, for at least 12 hours and up to 48 hours at 40° C. in a humidified incubator (Shake-N-Bake hybridization oven). Following hybridization, sections were washed 2× for 20 minutes each, agitated, in PBSTwR at room temperature (RT). A final stringency wash for 20 minutes in PBSTwR (with SSC supplemented to a final concentration of 4×) was performed at 37° C., agitated. Sections were briefly rinsed with PBSTwR at RT. Sections were incubated in ligation mixture composed of 0.2 U/μl T4 DNA Ligase, 1× T4 Ligase Buffer, 0.1 mg/ml BSA, 0.2 U/μl Superase RNAse Inhibitor, and H₂O. For some sections, ligations were performed using 7.5% PEG6000 to increase ligation speed. In this case, T4 DNA Ligase buffer was formulated without DTT, which is not necessary for the reaction and precipitates in PEG. Ligations were performed at RT, agitated, for 2 hours. Sections were then washed 2× for 15 minutes each with PBSTwR. Sections were then incubated in rolling-circle amplification (RCA) buffer at 30° C. For combinatorial sequencing, samples were incubated for 2 hours with agitation. For single-amplicon resolved sequential sequencing, samples were also incubated for 2 hours with agitation. For sequential sequencing with summated signals per cell, samples were incubated for at least 2 hours and up to 24 hours. RCA buffer was composed of 0.6 U/μl Phi29 DNA polymerase, 1× Phi29 DNA Polymerase buffer, 0.25 mM dNTP, 0.05 mg/ml BSA, 40 uM 5-(3-aminoallyl)-dUTP, 0.2 U/μl Superase RNAse inhibitor, and H₂O. Sections were subsequently washed 2× for 15 minutes each at RT, without agitation, in PBSTw (without RNAse inhibitor). If necessary, samples were placed at 4C until proceeding the following day. Samples were treated with 20 mM Acrylic Acid NHS Ester (AA-NHS) buffer for 2 hours at RT without agitation. Samples were rinsed 2× in PBSTw to replace AA-NHS buffer before being placed in pre-polymerization buffer (PM1) composed of 2×SSC, 4% Acrylamide monomer, 0.2% Bis-acrylamide monomer, and H₂O. Samples were incubated in PM1 at RT for 30 minutes to allow monomer to penetrate the section. Following incubation, PM1 solution was removed from the sections and replaced with 10-20 μl of ice-cold polymerization buffer (PM2), containing PM1 plus 1:1000 v/v Tetramethylethylenediamine (TEMED) and 1:1000 v/v ammonium persulfate (APS). PM2 was added to sections on ice. Coverslips coated with Gel Slick solution according to manufacturer's instructions were placed over sections and pressed down to remove air bubbles and minimize excess gel over the section. Sections were polymerized at RT for 1 to 1.5 hours. After removing coverslips with a pair of forceps and rinsing sections with PBSTw to remove unpolymerized monomer, sections were incubated for at least 2 hours and up to 24 hours in digestion buffer containing 2×SSC, 2% SDS, 0.8 mg/ml Proteinase K, 0.5% Triton X-100, and H₂O. Samples were finally washed 3× in PBS for 5 minutes each to remove digestion buffer, were stained for 10 minutes with DAPI at a concentration of 1:1000 and were subsequently rinsed with PBS. In some cases, the thin section samples were processed according to the following thick section protocol, except wash times were limited to 5-10 minutes per wash.

### STARmap2 Preparation—Thick Section, Combinatorial and Sequential

### Sample Preparation

Mice were transcardially perfused with ice-cold PBS, followed by ice-cold 4% PFA. Brains were dissected and transferred to ice-cold 4% PFA for overnight fixation with agitation, up to 24 hours. Brains were subsequently rinsed in ice-cold PBS and then equilibrated in ice-cold PBS at 4° C. for 30 minutes. Brains were sectioned in fresh, ice-cold PBS over ice on a vibratome to a thickness of between 50-300 m, with a typical thickness of 150 m. Sections containing the area of interest were transferred directly to ice-cold ethanol of 65% or greater, typically 70% ethanol in H₂O. Sections were stored until use at 4° C. in the ethanol solution for at least overnight and maximally for several months, though typically for less than a month.

### Library Generation

Sections were equilibrated to room temperature and rehydrated in PBSTwR for at least 30 minutes, followed by a second rinse with PBSTwR to remove any residual ethanol. Samples were placed into hybridization buffer containing 2× saline-sodium citrate (SSC) buffer, 10% formamide, 0.1 mg/mL sheared salmon sperm DNA, probe sets appropriate for combinatorial or sequential sequencing (see Targeted probes) at a final concentration of between 1 nM and 100 nM per probe pair (probe aliquot heated to 90° C. for 3 minutes and allowed to cool slowly to room temperature prior to addition to hybridization buffer), mRNA-label oligo at 3 μM, acrydite adaptor oligo (for gel retention of probe/RNA complexes) at one half to two-times equimolar concentration with the total concentration of probes, Superase RNAse inhibitor at a concentration of 0.2 U/μl, and RNAse-free distilled, deionized water (H₂O), and 10% sterile Dextran-sulfate solution. At least 125 μl of hybridization buffer was added to samples and typically 250 μl was used. Sections were incubated, agitated, for at least 12 hours and typically between 16 to 48 hours at 40° C. in a humidified incubator (Shake-N-Bake hybridization oven). Sections were washed 2× for 30 minutes each in PBSTwR supplemented to 2×SSC at 37° C., agitated. They were then washed at RT, agitated, in PBSTwR, for 30 minutes, and then were rinsed in PBSTwR. Buffer was replaced with degassed PM1 containing 3% acrylamide and 0.15% bis-acrylamide and sections were placed on ice for at least 1 hour to allow PM1 monomers to penetrate into the section. PM1 was subsequently removed and, with the samples still on ice, replaced with 40 μl of PM2 (prepared on ice) and coverslipped with a Gel-Slick coated coverslip. Coverslips were pressed down to remove air bubbles and ensure uniform gelation with minimal gel over the sections, and sections were placed to RT for gelation for 1.5 hours. In some cases, this gelation step was performed with sections in wells whose coverglass had been pre-treated according to manufacturer's instructions with Bind-Silane, such that gelation caused the sample to adhere to the well-plate glass, and subsequent library preparation steps were performed with the sample immobilized on the glass. In other cases, the gelation step was performed without treated glass, and in this case the rest of the sample preparation steps were performed with a floating section, allowing for better reagent diffusion (from both sides of the resulting gel) into the sample, better uniformity of resulting signals, and more efficient enzymatic action due to the increased diffusion. Following gelation, coverslips were removed with forceps and sections were rinsed with PBSTwR before being transferred to digestion buffer as described above and incubated for 16-24 hours at 37° C. with agitation to clear. In some cases samples were digested for 48 hours. Samples that did not appear optically transparent were further cleared in fresh buffer. After completing digestion, sections were washed either 3× for 30 min at RT agitating with PBSTwR, or 1× with PBS+2 mM PMSF solution for 30 min at RT agitating, to inactivate the proteinase K, and then 2× for 30 min at RT agitating with PBSTwR. Sections were incubated in ligation mixture composed of 0.2 U/μl T4 DNA Ligase, 1× T4 Ligase Buffer, 0.1 mg/ml BSA, 0.2 U/μl Superase RNAse Inhibitor, and H₂O. For some sections, ligations were performed using 7.5% PEG6000 to increase ligation speed. In this case, T4 DNA Ligase buffer was formulated without DTT, which is not necessary for the reaction and precipitates in PEG. Ligations were performed at RT, agitated, for 12-24 hours, typically 16 hours. Sections were then washed 2× for 30 minutes each with PBSTwR with agitation at RT. Samples were pre-incubated in RCA buffer at 4° C. for approximately 6 hours, composed of 0.6 U/μl Phi29 DNA polymerase, 1× Phi29 DNA Polymerase buffer, 0.25 mM dNTP, 0.05 mg/ml BSA, 40 μM 5-(3-aminoallyl)-dUTP, 0.2 U/μl Superase RNAse inhibitor, and H₂O. In some cases, including in thin section preparations as well as thick section, RCA buffer additionally contained amplicon condensing and detection oligo, which sometimes contained an end modification for attachment to the gel, such as a 5′ primary amine or Acrydite, and contained a unique sequence for amplicon detection in addition to two or more copies of a sequence complementary to a common sequence on the amplicons, plus one or more “N” bases positioned next to each of the complementary sequences. The common sequence on the amplicons may be positioned next to the sequence originally matching the probe target (for example, the endogenous mRNA), such that an amplicon condensing and detection oligo pool with randomized bases will tend to sort into oligos largely or completely matching amplicons of a specific gene. This has the effect of drawing together strands from a given amplicon for a particular gene (decreasing the optical spread of the amplicon) and making it less likely that adjacent amplicons from different genes will aggregate when densely packed. The preincubation of the sample in RCA mixture (optionally without dNTPs) at 4° C. allows the polymerase to penetrate the sample uniformly in Z while minimizing end degradation of the extended oligo or degradation of the polymerase due to instability. Following preincubation, the sample was transferred to 30° C. with rocking for 2 to 24 hours, typically 16 hours for sequentially encodings and 3 hours for combinatorial encodings, as the thicker sections are more challenging optically and require greater amplification of signals compared to thin sections. When condensing oligos are not used, a 3 hour incubation at 30° C. provides an optimal balance between signal intensity per amplicon and uniformity of amplicon shape. The amplicon condensing and detection oligo helps to ensure that increased amplification times do not overwhelm downstream computational detection of amplicons due to the growth in amplicon size or irregularity of amplicon shape, especially for combinatorial sequencing in thick samples. Following amplification, sections were washed 2× with PBS for 20 minutes at RT without agitation. For samples in which the previous steps were performed on floating sections (the majority of cases), the sections were subsequently attached to the glass bottom of plates. To do so, in most cases samples were transferred to poly-L-lysine coated glass-bottom wells and oriented as desired. Buffer was then totally removed and samples were allowed to somewhat dry-down flat onto the glass (for 10 minutes at room temperature). Samples were then soaked in excess cross-linker that attaches to amines, such as PFA, Glutaraldehyde, BSPEG9, or other Bis-NHS cross linkers (with intermediate chains of varying lengths and compositions) (typically with glutaraldehyde), for 1 hour at room temperature, before quenching with Tris for 30 minutes and final washing 3× with PBS for 20 minutes each at RT, with the second wash containing DAPI at 1:1000. In some cases, samples were additionally stabilized on the coverglass through the addition of a rapidly polymerizing elastomer agent around the border of the sample. In some cases, floating sections were incubated in AA-NHS buffer (as described in the thin section methods section) for two hours at room temperature, washed for 2×20 minutes in PBS at RT, and then incubated in degassed PM1 at RT for 30 minutes. Sections were then soaked on ice in PM2 for 5 minutes before being transferred to Bind-Silane treated glass-bottom wells on ice, where all PM2 solution was removed, samples were pressed flat against the well bottom, residual PM2 was drained with a Kimwipe (to prevent any excess gelation), samples were covered with a Gel-Slick coated coverglass, and subsequently were allowed to polymerize at RT for 1 hour, following which coverslips were removed and sections were washed 3× for 20 minutes with PBS at RT, with the second wash containing DAPI at 1:1000 concentration. For sections that were not floating, but were instead attached to the glass-bottom of the well during the first gelation, samples were treated with a cross-linker to better retain amplicons, such as Glutaraldehyde, PFA, BSPEG9, or other Bis-NHS cross linkers, quenched with Tris, and then washed, as described above. Finally, in some cases, the entire thick section procedure was performed on samples that had previously undergone hydrogel formation and clearing, in which splinted padlock probes and/or targets such as exogenous oligos (as barcodes or labeling other cellular targets), endogenous RNAs, or cellular components were retained into the hydrogel (ex: CLARITY procedure). The linkage of these probes or targets into the gel was performed as previously described for hydrogel attachment or by other chemical crosslinkers combined with hydrogel monomer moieties (for instance, by reacting alkylating agents like Melphalan with acrylic acid NHS ester or Acryloyl-X), specific antibodies, or oligos, modified such that these substrates were incorporated into the gel upon gel formation. The thick (or in some cases thin, in which case the final gelation step is omitted) section STARmap2 procedure was then performed starting from hybridization (in the case of non-splinted padlock probes), with the first gelation and digestion step removed, or from the ligation step.

### Sequencing Cycling—SCAL and SEDAL2

A sequencing cycle for SCAL or SEDAL2 optionally begins with a brief sample wash, before proceeding to the first signal addition. For SCAL sequencing, depending on whether sequential or combinatorial encoding is being used for a particular round, the corresponding set of OR and fluor oligos or RO and fluor oligos and their round-specific competitors are added to the sample in a ligase mixture, consisting of BSA (1:100), T4 Ligase (1:20), 10× T4 Ligase buffer (as described above, without DTT), PEG6000 (7.5%), DAPI (1:1000), and H₂O. In combinatorial encodings, the RO oligo for a given position x is added, plus the dibase-encoding fluorophore set of 16 oligos, plus the competitor oligo for the previous position that was labeled (unless it is the first round of labeling, in which case competitor oligo is omitted). In sequential encodings, OR oligo for a given round x, the 4-channel fluorophore mixture, and the round x-1 competitor oligo are added, except if it is the first round of labeling. The presence of PEG in the sequencing ligation mixture substantially accelerates the signal addition onto the target. Target is labeled for 1-4 hours in the labeling phase. Subsequently, the sample is washed in 1×SSC buffer to remove excess fluorophore and OR/RO oligo, before buffer is exchanged into imaging buffer, which consist of antifade solution. Antifade solution is a two-part solution, stored separately to maintain activity at room temperature for longer periods of time. The first component of imaging buffer contains Tris HCl (125 mM), SSC (1×), H₂O, Trolox-Quinone (TQ) solution (at ˜500 μM quinone in stock, 100 μM TQ in imaging buffer), 2 mM Trolox, Glucose oxidase (0.66 mg/ml), and Catalase (0.8 mg/ml). The second component of imaging buffer consists of 1.11 M glucose in 1×SSC. Following incubation of the sample in imaging buffer, the sample is imaged, and briefly rinsed before proceeding to the next sequencing cycle.

For SEDAL2, the same oligo/ligation mixture is used as described as above during the signal addition phase, except competitor oligos are omitted, and OR/RO oligos do not contain competitor-specific complimentary sequence in the oligo. Following sample addition, washing, imaging buffer addition, and imaging as described above, SEDAL2 includes a separate phase for signal removal, in which signals are either stripped off with formamide-containing stripping solution (ex: 80% formamide, 0.1% Triton-X, 2×SSC, and H₂O) or if thiol-linked dyes are used for sequential encoding fluorescent oligos, cleaving solution containing 2×SSC, 50 mM TCEP, and H₂O. Samples are subsequently washed in either 1×SSC in H₂O or PBSTw as described above, before proceeding to the next round of signal addition.

### Data Transfer

As large amounts of data can be produced by sequencing runs, separate system threads are run during sequencing that regularly check for completed data files (images, metadata, configurations) and streams them to either to a large storage buffer disk or to a cloud compute storage, where data is further processed.

What is claimed is:

1. A method for in situ gene sequencing of a target nucleic acid in a cell in an intact tissue, the method comprising:
(a) contacting a fixed and permeabilized intact tissue with at least a pair of oligonucleotide primers under conditions to allow for specific hybridization,
wherein the pair of primers comprise a first oligonucleotide and a second oligonucleotide;
wherein each of the first oligonucleotide and the second oligonucleotide comprises a first complementarity region, a second complementarity region sequence, and a third complementarity region; wherein the second oligonucleotide further comprises a barcode sequence;
wherein the first complementarity region of the first oligonucleotide is complementary to a first portion of the target nucleic acid, wherein the second complementarity region of the first oligonucleotide is complementary to the first complementarity region of the second oligonucleotide,
wherein the third complementarity region of the first oligonucleotide is complementary to the third complementarity region of the second oligonucleotide, wherein the second complementary region of the second oligonucleotide is complementary to a second portion of the target nucleic acid,
wherein the first portion of the target nucleic is adjacent to the second portion of the target nucleic acid, wherein the second complementarity region of the first oligonucleotide comprises a first portion of a unique matching sequence and the third complementarity region of the first oligonucleotide comprises a second portion of the unique matching sequence, and the first complementarity region of the second oligonucleotide comprises a sequence that is complementary to the first portion of the unique matching sequence, and the third complementarity region of the second oligonucleotide comprises a sequence that is complementary to the second portion of the unique matching sequence;
(b) adding ligase to ligate the second oligonucleotide and generate a closed nucleic acid circle;
(c) performing rolling circle amplification in the presence of a nucleic acid molecule, wherein the performing comprises using the second oligonucleotide as a template and the first oligonucleotide as a primer for a polymerase to form one or more amplicons;
(d) embedding the one or more amplicons in the presence of hydrogel subunits to form one or more hydrogel-embedded amplicons;
(e) contacting the one or more hydrogel-embedded amplicons having the barcode sequence with a set of sequencing primers under conditions to allow for ligation, wherein the set of sequencing primers comprises a third oligonucleotide configured to decode bases and a fourth oligonucleotide configured to convert decoded bases into a signal, wherein the ligation only occurs when both the third oligonucleotide and the fourth oligonucleotide are complementary to adjacent sequences of the same amplicon;
(f) reiterating step (e); and
(g) imaging the one or more hydrogel-embedded amplicons to determine in situ a gene sequence of the target nucleic acid in the cell in the intact tissue.

2. The method of claim 1 or 2, further comprising contacting the fixed and permeabilized intact tissue with a gel adaptor oligonucleotide that binds to the first oligonucleotide, wherein the gel adaptor oligonucleotide comprises a nucleotide modification at the 5′ end or the 3′ end that links the gel adapter to the hydrogel during gelation.

3. The method of claim 2, wherein the modification comprises an acrydite group.

4. The method of claim 2 or 3, wherein the first oligonucleotide further comprises a common binding site for the gel adaptor oligonucleotide.

5. The method of claim 4, wherein the common binding site for the gel adaptor oligonucleotide is adjacent to the first complementarity region of the first oligonucleotide.

6. The method of any one of claims 1-5, further comprising contacting the fixed and permeabilized intact tissue with an oligonucleotide probe for detection and condensing of the amplicons, wherein the oligonucleotide probe binds to the second oligonucleotide.

7. The method of claim 6, wherein the second oligonucleotide further comprises a common binding site for the oligonucleotide probe for detection and condensing of the amplicons.

8. The method of claim 7, wherein the common binding site for the oligonucleotide probe for detection and condensing of the amplicons is adjacent to the second complementarity region or adjacent to the sequence that is complementary to the second half of the unique matching sequence of the second oligonucleotide.

9. The method of any one of claims 6-8, wherein the oligonucleotide probe comprises a unique sequence for detecting an amplicon of a probe target of interest and two or more copies of a sequence complementary to a common sequence on the amplicons.

10. The method of any one of claims 6-9, wherein the oligonucleotide probe further comprises a nucleotide modification at the 5′ end or the 3′ end such that the first oligonucleotide probe is linked to the hydrogel during gelation.

11. The method of claim 10, wherein the modification comprises an acrydite group.

12. The method of any one of claims 1-11, further comprising barcoding a cell by contacting the cell with:
a first probe comprising a 5′-amine modification or a 5′-biotin modification, a common gel adaptor complementary sequence that hybridizes with the gel adaptor oligonucleotide, and a unique barcode sequence; and
a second probe comprising a first sequence that is complementary to a first portion of the unique barcode sequence and a second sequence that is complementary to a second portion of the unique barcode sequence, wherein the first sequence and the second sequence flank a sequencing encoding sequence, wherein hybridization of the first probe and the second probe results in formation of a barcoding complex comprising the first probe and the second probe.

13. The method of claim 12, wherein the second probe is a padlock probe.

14. The method of any one of claims 1-13, wherein the first portion of the target nucleic acid and the second portion of the target nucleic acid have about the same melting temperature.

15. The method of any one of claims 1-14, further comprising contacting the fixed and permeabilized intact tissue with an mRNA retention oligonucleotide, wherein the mRNA retention oligonucleotide comprises:
a nucleotide modification at the 5′ end or the 3′ end, wherein the mRNA retention oligonucleotide is linked to the hydrogel during gelation;
a poly-T tail that hybridizes to a poly-A tail of an mRNA, wherein hybridization of the poly-T tail of the mRNA retention oligonucleotide to the poly-A tail of the mRNA retains the mRNA in the hydrogel; and
a unique hybridization sequence.

16. The method of claim 15, wherein the poly-T tail comprises interleaved locked nucleic acid (LNA) thymine (T) bases.

17. The method of claim 15 or 16, further comprising contacting the fixed and permeabilized intact tissue with a fluorescently labeled probe oligonucleotide that selectively binds to the unique hybridization sequence of the mRNA retention oligonucleotide.

18. The method of any one of claims 1-17, wherein sequencing is performed with sequential or combinatorial encoding.

19. The method of any one of claims 1-18, further comprising preincubating the tissue sample with the polymerase for a sufficient time to allow uniform diffusion of the polymerase throughout the tissue before performing the rolling circle amplification.

20. The method of any one of claims 1-19, wherein said imaging is performed in presence of an anti-fade buffer comprising an antioxidant.

21. The method of claim 20, wherein the anti-fade buffer comprises trolox (6-hydroxy-2,5,7,8-tetramethylchroman-2-carboxylic acid) and trolox-quinone.

22. The method of any one of claims 1-21, wherein the signal is a fluorescent signal.

23. The method of claim 22, further comprising removing the signal after imaging by contacting the hydrogel with formamide.

24. The method of claim 22, wherein the fourth oligonucleotide is covalently linked to a fluorophore by a disulfide bond.

25. The method of claim 24, further comprising contacting the hydrogel with a reducing agent after said imaging, wherein reduction of the disulfide bond results in cleavage of the fluorophore from the fourth oligonucleotide.

26. The method of any one of claims 1-25, wherein the set of primers are denatured by heating before contacting the sample.

27. The method of any one of claims 1-26, wherein the cell is present in a population of cells.

28. The method of claim 27, wherein the population of cells comprises a plurality of cell types.

29. The method of any one of claims 1-28, wherein the contacting the fixed and permeabilized intact tissue comprises hybridizing the primers to the same target nucleic acid.

30. The method of any one of claims 1-29, wherein the target nucleic acid is RNA or DNA.

31. The method of claim 30, wherein the RNA is mRNA.

32. The method of any one of claims 1-31, wherein the second oligonucleotide comprises a padlock probe.

33. The method of any one of claims 1-32, wherein the first complementarity region of the first oligonucleotide has a length of 19-25 nucleotides.

34. The method of any one of claims 1-33, wherein the second complementarity region of the first oligonucleotide has a length of 6 nucleotides.

35. The method of any one of claims 1-34, wherein the third complementarity region of the first oligonucleotide has a length of 6 nucleotides.

36. The method of any one of claims 1-35, wherein the first complementarity region of the second oligonucleotide has a length of 6 nucleotides.

37. The method of any one of claims 1-36, wherein the second complementarity region of the second oligonucleotide has a length of 19-25 nucleotides.

38. The method of any one of claims 1-37, wherein the third complementarity region of the second oligonucleotide has a length of 6 nucleotides.

39. The method of any one of claims 1-38, wherein the first complementarity region of the second oligonucleotide comprises the 5′ end of the second oligonucleotide.

40. The method of any one of claims 1-39, wherein the third complementarity region of the second oligonucleotide comprises the 3′ end of the second oligonucleotide.

41. The method of any one of claims 1-40, wherein the first complementarity region of the second oligonucleotide is adjacent to the third complementarity region of the second oligonucleotide.

42. The method of any one of claims 1-41, wherein the barcode sequence of the second oligonucleotide provides barcoding information for identification of the target nucleic acid.

43. The method of any one of claims 1-42, wherein the contacting the fixed and permeabilized intact tissue comprises hybridizing a plurality of oligonucleotide primers having specificity for different target nucleic acids.

44. The method of any one of claims 1-43, wherein the second oligonucleotide is provided as a closed nucleic acid circle, and the step of adding ligase is omitted.

45. The method of any of claims 1-44, wherein the melting temperature (Tₘ) of oligonucleotides is selected to minimize ligation in solution.

46. The method of any one of claims 1-45, wherein the adding ligase comprises adding a DNA ligase.

47. The method of any one of claims 1-46, wherein the nucleic acid molecule comprises an amine-modified nucleotide.

48. The method of claim 47, wherein the amine-modified nucleotide comprises an acrylic acid N-hydroxysuccinimide moiety modification.

49. The method of any one of claims 1-48, wherein the embedding comprises copolymerizing the one or more amplicons with acrylamide.

50. The method of any one of claims 1-49, wherein the embedding comprises clearing the one or more hydrogel-embedded amplicons wherein the target nucleic acid is substantially retained in the one or more hydrogel-embedded amplicons.

51. The method of claim 50, wherein the clearing comprises substantially removing a plurality of cellular components from the one or more hydrogel-embedded amplicons.

52. The method of claim 50 or 51, wherein the clearing comprises substantially removing lipids or proteins, or a combination thereof from the one or more hydrogel-embedded amplicons.

53. The method of any one of claims 1-52, wherein the contacting the one or more hydrogel-embedded amplicons comprises eliminating error accumulation as sequencing proceeds.

54. The method of any one of claims 1-53, wherein the imaging comprises imaging the one or more hydrogel-embedded amplicons using confocal microscopy, two-photon microscopy, light-field microscopy, intact tissue expansion microscopy, and/or CLARITY™-optimized light sheet microscopy (COLM).

55. The method of any one of claims 1-54, wherein the intact tissue is a thin slice.

56. The method of claim 55, wherein the intact tissue has a thickness of 5-20 μm.

57. The method of claim 55 or 56, wherein the contacting the one or more hydrogel-embedded amplicons occurs four times or more.

58. The method of any one of claims 1-54, wherein the intact tissue is a thick slice.

59. The method of claim 58, wherein the intact tissue has a thickness of 50-200 μm.

60. The method of claim 58 or 59, wherein the contacting the one or more hydrogel-embedded amplicons occurs six times or more.

61. The method of any one of claims 1-60, wherein the ligation of the third oligonucleotide and the fourth oligonucleotide is performed in presence of a polyethylene glycol polymer.

62. The method of claim 61, wherein the PEG polymer is PEG 6000.

63. The method of any one of claims 1-62, wherein the unique matching sequence is a randomized sequence.

64. A method of screening a candidate agent to determine whether the candidate agent modulates gene expression of a nucleic acid in a cell in an intact tissue, the method comprising performing the method of any one of claims 1-63 to determine the gene sequence of the target nucleic acid in the cell in the intact tissue, and
detecting the level of gene expression of the target nucleic acid, wherein an alteration in the level of expression of the target nucleic acid in the presence of the candidate agent relative to the level of expression of the target nucleic acid in the absence of the candidate agent indicates that the candidate agent modulates gene expression of the nucleic acid in the cell in the intact tissue.

65. The method of claim 64, wherein the detecting comprises performing flow cytometry; sequencing; probe binding and electrochemical detection; pH alteration; catalysis induced by enzymes bound to DNA tags; quantum entanglement; Raman spectroscopy; terahertz wave technology; and/or scanning electron microscopy.

66. The method of claim 65, wherein the flow cytometry is mass cytometry or fluorescence-activated flow cytometry.

67. The method of any one of claims 64-66, wherein the detecting comprises performing microscopy, scanning mass spectrometry, or other imaging techniques

68. The method of any one of claims 64-67, wherein the detecting comprises detecting a signal.

69. The method of claim 68, wherein the signal is a fluorescent signal.

70. A system, comprising:
a fluidics device, and
a processor unit configured to perform the method of any one of claims 1-69.

71. The system of claim 70, further comprising an imaging chamber.

72. The system of claim 70 or 71, further comprising a pump.
